(19) United States
(12) Patent Application Publication
(10) Pub. No.: US 2010/0048657 A1
(43) Pub. Date: Feb. 25, 2010

(54) CONTROL OF MALIGNANT CELLS PROLIFERATION THROUGH THE INHIBITION OF CASEIN KINASE 2

(75) Inventor: Michael Kalafatis, Shaker Heights, OH (US)

Correspondence Address: RANKIN, HILL & CLARK LLP, 38210 Glenn Avenue, WILLOUGHBY, OH, 44094-7808, US

(73) Assignee: CLEVELAND STATE UNIVERSITY, Cleveland, OH (US)

(21) Appl. No.: 12/441,077

(22) PCT Filed: Sep. 11, 2007
(86) PCT No.: PCT/US07/19676

§ 371 Date: Aug. 4, 2009

Related U.S. Application Data
(60) Provisional application No. 60/844,022, filed on Sep. 12, 2006.

(51) Int. Cl. A61K 31/4184; A61K 31/4192; A61P 35/02

(52) U.S. Cl. 514/393; 514/359

(57) ABSTRACT

Methods and related compositions are disclosed for treating an array of myeloproliferative disorders and hematological malignancies. In particular, treatment methods and compositions for treating chronic myelogenous leukemia are disclosed. The methods and compositions utilize certain casein kinases, and specifically CK2α agents.

This application claims priority from U.S. provisional application Ser. No. 60/844,022 filed Sep. 12, 2006, herein incorporated by reference.

### BACKGROUND

The present invention relates to methods and related compositions for treating myeloproliferative disorders such as chronic myelogenous leukemia (CML). The invention also relates to inhibiting hematological malignancies, inducing maturation of malignant megakaryoblasts, inducing thrombocytosis, reducing platelet production otherwise occurring from malignant megakaryoblasts, and methods for inducing thrombocytopolesis. The present invention finds particular application in conjunction with treating CML, and will be described with particular reference thereto. However, it is to be appreciated that the present invention is also amenable to other like applications.

Cancer cells are characterized by increased proliferation and loss of the normal phenotype and function. Cancer is caused by defects in signaling pathways including deregulation of apoptosis process. Apoptosis is a genetically programmed and evolutionary conserved mechanism through which the normal development and tissue homeostasis are maintained (Borgers et al., 2000; Fadeel et al., 1999; Ishizaki et al., 1995; Leist and Jaattela, 2001; Nilsson and Cleveland, 2003; Saraste and Pulkki, 2000). Full citation of background articles is set forth at the end of the Detailed Description.

Megakaryocytes are polyploid cells, originating from hematopoietic stem cells in the bone marrow. Thrombocytopoiesis is the process of production of anucleated cells, platelets, from megakaryocytes (Italiano J R. and Shivdasani, 2003). Megakaryoblasts undergo endomitosis and maturation to the stage of megakaryocytes, through a process called megakaryocytopoiesis (Gewirtz, 1995). Proplatelets bearing megakaryocytes fragment to give rise to platelets, through the process of thrombocytopoiesis. Platelets (thrombocytes) are vital for maintaining normal hemostasis and for the response of the body to trauma. Platelets formation process is complex and not well understood (Patel et al., 2005). The thrombocytopoiesis process has been linked with constitutive apoptosis of megakaryocytes. Caspases activation in megakaryocytes has also been connected with the platelets production. Pro-apoptotic and pro-survival proteins are regulated towards apoptosis during megakaryocytopoiesis and thrombocytopoiesis (Clarke et al., 2003; De Botton et al., 2002; Italiano J R. and Shivdasani, 2003; Kaluzhny and Ravid, 2004; Kaluzhny et al., 2002; Mazur, 1987; Zunino et al., 2001).

The reciprocal chromosomal translocation t(9;22), known as the Philadelphia positive chromosome (Ph+) (Rowley, 1973) is associated with diseases like chronic myelogenous leukemia (CML), acute myelogenous leukemia (AML), acute non-lymphocytic leukemia (ANLL) and acute lymphocytic leukemia (ALL). This genetic abnormality results in the chimeric oncoprotein BCR/ABL tyrosine kinase, which is thought to be the main cause of the abnormal survival and over-proliferation of hematopoietic stem cells and their progeny (Abelson and Rabstein, 1970; Druker et al., 2002; McGahon et al., 1994). In chronic myelogenous leukemia, the BCR/ABL tyrosine kinase is constitutively activated. Different intracellular pathways are transformed by the oncoprotein BCR/ABL, resulting in uncontrolled hematopoietic proliferation. The late phase of chronic myelogenous leukemia, named blast crisis (or blastic phase), is characterized by extreme overproliferation of stem cells and their progeny in bone marrow. In blast crisis, a major complication is thrombosis due to high platelet counts (Deininger and Druker, 2003; Druker et al., 2002). Thrombosis is lethal because it leads to organ damage (stroke, hearth attack). In myeloproliferative disorders, like chronic myelogenous leukemia, the platelet counts and function are abnormal due to malignant megakaryoblasts overproliferation.

In different cancers, including leukemias, a tyrosine kinase named casein kinase 2 (CK2) was found to be constitutively activated, elevated and to serve as an oncoprotein (Izeradjene et al., 2004; Izeradjene et al., 2005; Landesman-Bollag et al., 2001; Piazza et al., 2006; Seldin et al., 2005; Slaton et al., 2004; Unger et al., 2004; Wang et al., 2001). CK2 is a plelotropic, ubiquitous ectokinase that phosphorylates Ser, Thr amino acid residues. The protein is a heterotetramer with two catalytic subunits, α and α′, and two regulatory β subunits (Glover et al., 1983; Niefind et al., 2001; Padmanabha and Glover, 1987; Pechkova et al., 2003; Pinna, 1990; Pinna, 1997; Pinna, 2002; Pinna, 2003; Schmidt-Spaniol et al., 1993; Stigare et al., 1993). Each subunit was shown to be able to execute specific functions by itself or in the holoenzyme form, the αα′β2 tetramer (Graham and Litchfield, 2000; Schmidt-Spaniol et al., 1993; Stigare et al., 1993). The up regulation and hyperactivity of CK2 has an anti-apoptotic function in leukemias (acute myelogenous leukemia, chronic myelogenous leukemia). This results in abnormal platelets counts and function (Phan-Dinh-Tuy et al., 1985). Interestingly, CK2α was found to be a substrate for the ABL domain of BCR/ABL (Hériché and Chambaz, 1998) and to form a specific complex with the BCR domain of BCR/ABL (Mishra et al., 2003). It was hypothesized that CK2α impedes sterically the binding of the ABL SH2 domain to BCR (Mishra et al., 2003). This results in proliferation abnormalities in Philadelphia positive cells. Therefore CK2α was shown to be a possible arbitrator of BCR/ABL function (Hériché and Chambaz, 1998; Mishra et al., 2003). Other functions of CK2α downstream of the BCR/ABL interaction give an overall oncogenic response in Philadelphia positive cells.

CK2α protein kinase inhibitors have been developed and studied, like Emodin; 5,6-dichloro-1-beta-D-ribofuranosylbenzimidazole (DRB); 4,5,6,7-Tetrabromobenzotriazole (TBB); 2-Dimethylamino-4,5,6,7-tetrabromo-1H-benzimidazole (DMAT); and ellagic acid (Battistutta et al., 2005; Cozza et al., 2006; Pagano et al., 2004a; Pagano et al., 2004b; Ruzzene et al., 2002; Sarno et al., 2005a; Sarno et al., 2005b). The inhibition of CK2 in various cancer cell lines produced apoptosis and proliferation arrest (Mishra et al., 2006; Pagano et al., 2004a; Pagano et al., 2006; Pagano et al., 2004b; Piazza et al., 2006; Pinna, 1990; Pinna, 1997; Pinna, 2002; Pinna, 2003; Ruzzene et al., 2002; Schmidt-Spaniol et al., 1993; Seldin et al., 2005; Slaton et al., 2004; Wang et al., 2001).

MEG-01 cells have previously been isolated from a patient with CML, Ph+, in blast crisis, with high peripheral blast counts and thrombocytosis (high platelets counts) (Ogura et al., 1985). The cells were characterized as being megakaryoblasts in an early stage of differentiation in the megakaryocytic lineage. The cells present the integrin αIIbβ3 expressed on their surface and are positive for platelet peroxidase (Ogura et al., 1985; Ogura et al., 1988). MEG-01 cells are Ph+, and express the p210 Bcr/Abl tyrosine kinase (Wertheim et al., 2002). MEG-01 cells were found to be cytokine independent (Lionberger et al., 2000; Liu et al., 1999). MEG-01 cells are capable of differentiating in vitro in response to PMA (Baatout, 1998), nitric oxide (NO) (Battinelli et al., 2001), aphidicolin, nocodazole (apoptosis inducers, by inhibiting DNA synthesis) and staurosporine (Ogura et al., 1988; Takeuchi et al., 1991; Takeuchi et al., 1998; Yamazaki et al., 1999). MEG-01 cells were found to release platelet-like particles following these treatments. Caspases inhibition in MEG-01 cell line was impeding proplatelet formation and platelets release (Clarke et al., 2003; De Botton et al., 2002).

Although extensive efforts have been directed toward the treatment of myeloproliferative disorders, a need remains for an improved treatment method. In particular, it would be desirable to provide an improved treatment strategy and related pharmaceutical formulations for the treatment of chronic myelogenous leukemia (CML).

Similarly, although significant research has been directed toward various hematogical malignancies, a need remains for improved strategies and related formulations for their treatment. In particular, a need exists for regimes for inhibiting hematological malignancies, inducing maturation of malignant megakaryoblasts, inducing thrombocytosis, reducing platelet production otherwise occurring from malignant megakaryoblasts, and methods for inducing thrombocytopoiesis.

### BRIEF DESCRIPTION

The present invention provides, in one aspect, a method for treating myeloproliferative disorders. The method comprises selectively inhibiting CK2α activity.

In another aspect, the present invention provides a method for inhibiting hematological malignancies. The method comprises selectively inhibiting CK2α activity.

In still another aspect, the present invention provides a method for inducing maturation of malignant megakaryoblasts. The method comprises selectively inhibiting CK2α activity.

In yet another aspect, the present invention provides a method for inducing thrombocytosis by selectively inhibiting CK2α activity.

In another aspect, the present invention provides a method for reducing platelet production occurring from malignant megakaryoblasts. The method comprises selectively inhibiting CK2α activity.

The present invention also provides a method for inducing thrombocytopolesis by selectively inhibiting CK2α activity.

In another aspect, the present invention provides a pharmaceutical composition comprising a CK2α inhibitor selected from the group consisting of (i) 4,5,6,7-tetrabromobenzotriazole (TBBt), (ii) 2-Dimethylamino-4,5,6,7-tetrabromo-1H-benzimidazole (DMAT), and combinations of (i) and (ii); and a pharmaceutically acceptable carrier.

### BRIEF DESCRIPTION OF THE DRAWINGS

FIG. 1 is a graph illustrating the effect of two preferred embodiment inhibitors in accordance with the present invention.

FIG. 2 is a photograph illustrating the area and number of malignant colonies in an untreated cell line.

FIG. 3 is a photograph illustrating the significantly reduced area and absence of any colonies in a cell line treated in accordance with the present invention.

FIG. 4 is a graph illustrating colony areas in 30 images of cells in an untreated control cell line, and a corresponding cell line treated with a preferred embodiment inhibitor in accordance with the present invention.

FIG. 5 is a graph illustrating percentages of apoptotic cells after 24 hours using two preferred embodiment inhibitors.

FIG. 6 is a graph illustrating percentages of apoptotic cells after 72 hours using two preferred embodiment inhibitors.

FIG. 7 is a graph illustrating percentages of apoptotic cells after 96 hours using two preferred embodiment inhibitors.

FIG. 8 is a graph or dot plot showing quadrant gating for a control cell line treated with both PI and Annexin V-FITC after 24 hours.

FIG. 9 is a dot plot showing quadrant gating for a cell line treated with a preferred embodiment inhibitor after 24 hours.

FIG. 10 is a photograph illustrating the effects of a preferred embodiment inhibitor upon a cell line, including the formation of proplatelet extensions.

FIG. 11 is a photograph illustrating the effects of a preferred embodiment inhibitor upon a cell line, including the formation of proplatelet extensions.

FIG. 12 is a photograph illustrating the effects of treating a cell line with a preferred embodiment inhibitor.

FIG. 13 is a photograph illustrating the effects of treating a cell line with a preferred embodiment inhibitor.

FIG. 14 is a photograph illustrating an untreated control cell line.

FIG. 15 is a phase contrast micrograph of cells treated with a preferred embodiment inhibitor.

FIG. 16 is a graph illustrating how treatment with a preferred inhibitor affects maturation of a cell line.

FIG. 17 is a graph illustrating how treatment with a preferred inhibitor affects maturation of a cell line.

FIG. 18 is a graph comparing the effects of two preferred embodiment inhibitors.

FIG. 19 is a graph illustrating maturation of a control cell line compared to a treated cell line.

FIG. 20 is a graph of a DNA content assay illustrating cells treated with a preferred embodiment inhibitor.

FIG. 21 is a photograph illustrating cells treated with a preferred embodiment inhibitor.

FIG. 22 is a photograph illustrating cells treated with a preferred embodiment inhibitor.

FIG. 23 is a photograph illustrating cells treated with a preferred embodiment inhibitor.

FIG. 24 is a graph illustrating cells treated with a preferred embodiment inhibitor.

FIG. 25 is a graph illustrating cells treated with a preferred embodiment inhibitor.

FIG. 26 is a graph illustrating cells treated with a preferred embodiment inhibitor.

FIG. 27 is a graph illustrating cells treated with a preferred embodiment inhibitor.

FIG. 28 is a graph illustrating cells treated with a preferred embodiment inhibitor.

FIG. 29 is an SEM image illustrating a clot comprising platelets produced from cells resulting from treatment with a preferred embodiment inhibitor.

FIG. 30 is an SEM image illustrating a clot comprising platelets produced from cells resulting from treatment with a preferred embodiment inhibitor.

FIG. 31 is an SEM image illustrating a clot comprising platelets produced from cells resulting from treatment with a preferred embodiment inhibitor.

### DETAILED DESCRIPTION

Megakaryoblasts are precursors of platelets that first differentiate to the stage of megakaryocytes. Mature megakaryocytes form pseudopodia and give rise to platelets. As described herein, the effect of casein kinase 2 alpha subunit (CK2α) inhibition was investigated with specific inhibitors in a megakaryoblastic cell line from a CML patient in blast crisis (MEG-01). It was surprisingly discovered that these inhibitors induce proliferation arrest while maintaining a steady cell number for a period of one week. Treated cells grew at a lower and constant rate than the non-treated cells. Apoptosis of MEG-01 was induced by CK2 inhibitors, and this phenomenon was found to be dose and time dependent. No necrosis was detected in the presence of the inhibitors, demonstrating that such compounds are not cytotoxic. In the presence of CK2 inhibitors, megakaryocytes matured to the pro-platelets bearing stage. Platelets were released through rupture, following cytoplasmic fragmentation and nuclear extrusion. Thrombocytopoiesis due to the use of CK2 inhibitors occurred both in suspension as well as with MEG-01 cells grown on a fibronectin matrix. Platelets obtained following these treatments were normal and were found to undergo shape change in response to various agonists (human thrombin, TRAP, ADP, PMA, fibronectin). These findings suggest that CK2 is involved in MEG-01 differentiation and platelets production and activation. Thus, by using a CK2 inhibitor, the present invention treatment strategy successfully stopped the abnormal proliferation of a transformed cell line and reversed the path towards its normal function.

The findings described in detail herein, demonstrate for the first time that CK2α inhibition with certain kinase inhibitors, induces malignant MEG-01 megakaryoblasts maturation and enhances functional platelet-like progeny release from MEG-01 cells. Interestingly, it was found that CKα inhibition resulting from treatment using either or both of the preferred embodiment inhibitors DMAT and TBB, induces proliferation arrest and apoptosis, without being cytotoxic. Proliferation arrest as well as induced apoptosis was correlated with the length and amount of treatment. DMAT, which in certain respects is a superior inhibitor than TBB (Kᵢ of 40 nM versus 400 nM), had effect at concentrations as little as 5 and 10 μM, and the concentration required for TBB to have the same effect like DMAT was at least two folds higher (50 μM). However, both inhibitors are believed to offer significant advance and benefit in the art.

Another striking result from the present invention is that CK2α inhibition with DMAT and TBB induced thrombocytopoiesis. The megakaryocytes undergoing thrombocytopoiesis showed apoptotic features, DNA condensation and fragmentation, blebbing and phosphatidylserine exposure. Mature megakaryocytes, of 100-150 μm in diameter, polyploid (as observed by DAPI staining) start to bleb and form pseudopods. Following explosive fragmentation, long filaments with beaded ends (proplatelets) are formed. The proplatelets do not stain positive in DAPI, showing that the beaded ends indeed will become platelets. Platelets are expelled out from the proplatelets, and the fragmented nucleus slowly is extruded. Thrombocytopoiesis process occurred in cells bound to fibronectin matrix as well on suspension cells.

The thrombocytopoiesis process follows the maturation and differentiation process of MEG-01 megakaryoblasts. This differentiation induction is similar to phorbol ester (PMA) effect, with the difference that CK2α inhibition is not cytotoxic, whereas PMA is a potent tumorigenic substance as well as a powerful platelet activator. This suggests involvement of various pathways and complex molecular interactions, besides the apoptotic machinery. Although not wishing to be bound to any particular theory, it is believed that the maturation of MEG-01 cells is a result of proliferation arrest that makes incomplete repeated cell cycle to enter into endomitosis, probably due to the action of CK2 on cell cycle. This hypothesis is also sustained by the preponderant nuclear localization of CK2α in malignant cells. This maturation process was assessed by the means of flow cytometric immunophenotyping (using an RPE conjugated monoclonal antibody, CD41a, and fibrinogen labeled with Alexa Fluor), considering the αIIbβ3 integrin maturation marker. αIIbβ3 integrin (which is also the fibrinogen receptor) increase in expression is correlated with increase in size and differentiation of megakaryoblasts. Increase in DNA content (DAPI staining) and cell size (as seen with light microscopy technique) demonstrates that MEG-01 cells mature due to CK2α inhibitor treatments.

The results described herein connect apoptosis with thrombocytopoiesis that follow megakaryocytopoiesis. It has been previously shown that platelet shedding results from a constitutive form of apoptosis of megakaryocytes (Battinelli et al., 2001; Clarke et al., 2003; De Botton et al., 2002; Italiano J R. and Shivdasani, 2003; Kaluzhny and Ravid, 2004; Kaluzhny et al., 2002; Mazur, 1987; Takeuchi et al., 1991; Takeuchi et al., 1998; Zauli et al., 1997; Zunino et al., 2001). However, the present invention demonstrates for the first time that CK2 inhibition induces platelets release from malignant megakaryoblast. For megakaryoblasts, CK2 inhibition first produces proliferation arrest, followed by differentiation to megakaryocytes that culminate with proplatelets formation, blabbing, and compartmentalized fragmentation of megakaryocytes, finalized by thrombocytes release.

Anchorage independence assays in soft agar show that CK2α inhibition with DMAT results in the repressing of malignant transformation of the MEG-01 cells. These results suggest that adherence pathways controlled by CK2α may be involved in the process. BCR/ABL was found to be involved in the malignant transformation of Ph+ cells, but its inhibition is not sufficient to suppress anchorage independence of such cells, suggesting involvement of other molecular mechanisms.

The platelets obtained in culture, following CK2α inhibition with specific kinase inhibitors are functional. These platelets form a clot visible with the eye (a normal fibrin clot as seen by SEM), when exposed to agonists.

In accordance with the present invention, CK2α inhibition studies with TBB and DMAT, demonstrate a key role of CK2 in oncogenic development as well as in the megakaryocytopoiesis and thrombocytopoiesis processes. This opens up the possibility of CK2 targeting drug design for patients with cytokine and BCR/ABL inhibitors resistance.

Due to the importance of protein kinases in malignant processes, the present invention has significant future therapeutic interest. The inventors were interested in determining the effect of CK2α inhibition on malignant megakaryoblast, with specific inhibitors. For this, the MEG-01 cell line was selected, characterized as being early stage megakaryoblasts with Philadelphia positive chromosome, isolated from a patient with CML, in blast crisis. These cells are extremely malignant with an increased proliferation rate.

The preferred embodiment CK2α inhibitors are DMAT and TBB. DMAT is 2-dimethylamino-4,5,6,7-tetrabromo-1H-benzimidazole and has the following structural formula (1):

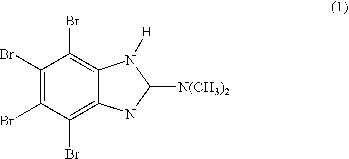

TBB (or sometimes referred to as TBBt herein) is 4,5,6,7-tetrabromobenzotriazole and has the following structural formula (2):

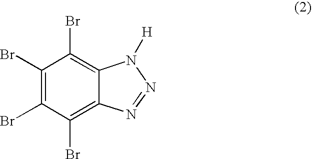

The present invention includes the use of either or both of the inhibitors DMAT and TBB, and/or their pharmaceutically acceptable salts. The preferred inhibitors can be incorporated into a wide array of compositions, formulations, and pharmaceuticals.

The pharmaceutical compositions may include an inhibitor by itself, or in combination and optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, alpha-lactose, anhydrous lactose, cellulose, sucrose, dextrose, sorbitol, modified dextrans, gum acacia, and starch. Some commercially available diluents are Fast-Flo, Emdex, STA-Rx 1500, Emcompress and Avicell. Such compositions may influence the physical state, stability, rate of in vivo release, and rate of in vivo clearance of the inhibitor compounds, see, e.g., Remington's Pharmaceutical Sciences, 18th Ed. pp. 1435-1712 (1990).

Pharmaceutically acceptable fillers can include, for example, lactose, microcrystalline cellulose, dicalcium phosphate, tricalcium phosphate, calcium sulfate, dextrose, mannitol, and/or sucrose. Inorganic salts including calcium triphosphate, magnesium carbonate, and sodium chloride may also be used as fillers in the pharmaceutical compositions. Amino acids may be used such as used in a buffer formulation of the pharmaceutical compositions.

Disintegrants may be included in solid dosage formulations of the inhibitors of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the composition, formulation, or pharmaceutical together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the composition, formulation, or pharmaceutical to prevent sticking during the formulation process. Lubricants may be used as a layer between the ingredients and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the composition, formulation, or pharmaceutical during formulation and to aid rearrangement during compression might be added. Suitable glidants include starch, talc, pyrogenic silica and hydrated silicoaluminate.

To aid dissolution of the composition, formulation, or pharmaceutical into an aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulations may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

In the methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded under the splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions as for example, proteinoid microspheres reported in U.S. Pat. No. 4,925,673. Liposomal encapsulation may include liposomes that are derivatized with various polymers, e.g., U.S. Pat. No. 5,013,556. In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about 1 mm. The formulation of the material for capsule administration could also be as a powder, lightly compressed plugs or even as tablets. The capsules could be prepared by compression.

The preferred embodiment inhibitors DMAT and TBB can be used and administered in a variety of forms, vehicles, and concentrations. Generally, the preferred embodiment inhibitors are used in conjunction with a vehicle such as DMSO, however a wide array of other vehicles may be employed. The inhibitor DMAT can be used so as to achieve in vivo or ex vivo concentrations in the vicinity of the cells of interest, ranging from as low as 0.1 μM to as high as 1,000 μM or more, however a preferred concentration range is from about 1 μM to about 100 μM and more preferably, from about 10 μM to about 50 μM. Similarly, the inhibitor TBB can be used so as to achieve in vivo or ex vivo concentrations in the vicinity of the cells of interest, ranging from as low as 0.1 μM to as high as 1,000 μM or more, however a preferred concentration range is from about 1 μM to about 150 μM and more preferably, from about 15 μM to about 75 μM. Generally, these concentrations are designated as effective amounts.

The instant pharmaceutical composition will generally contain a per dosage unit (e.g., tablet, capsule, powder, injection, teaspoonful and the like) from about 0.001 to about 100 mg/kg. In one embodiment, the instant pharmaceutical composition contains a per dosage unit of from about 0.01 to about 50 mg/kg of compound, and preferably from about 0.05 to about 20 mg/kg. Methods are known in the art for determining therapeutically effective doses for the instant pharmaceutical composition. The therapeutically effective amount for administering the pharmaceutical composition to a human, for example, can be determined mathematically from the results of animal studies.

The present invention provides methods for treating myeloproliferative disorders, and in particular, for treating chronic myelogenous leukemia. In a preferred treatment method, an effective amount of one or more preferred CK2α inhibitor(s) is administered to a subject for a duration sufficient to induce proliferation arrest while maintaining a steady cell number. Preferably, the duration ranges from about 1 to about 14 days, and more preferably from about 3 to about 7 days. The one or more preferred inhibitor(s) can be administered multiple times per day so as to produce a preferred effective amount. In addition, it is preferred that prolonged treatment strategies can be defined in accordance with the present invention.

The present invention also provides methods for treating various hematological malignancies, and in particular, for inhibiting hematological malignancies, inducing maturation of malignant megakaryoblasts, inducing thrombocytosis, reducing platelet production otherwise occurring from malignant megakaryoblasts, and methods for inducing thrombocytopoiesis. In a preferred treatment method, an effective amount of one or more preferred CK2α inhibitor(s) is administered to a subject for a duration sufficient to induce thrombocytopoiesis. Preferably, the duration ranges from about 1 to about 14 days, and more preferably from about 3 to about 7 days. The one or more preferred inhibitor(s) can be administered multiple times per day so as to produce a preferred effective amount. In addition, it is preferred that prolonged treatment strategies can be defined in accordance with the present invention.

The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage, see, for example, Remington's Pharmaceutical Sciences, pp. 1435-1712. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various indications involving aberrant proliferation of hematopoletic cells.

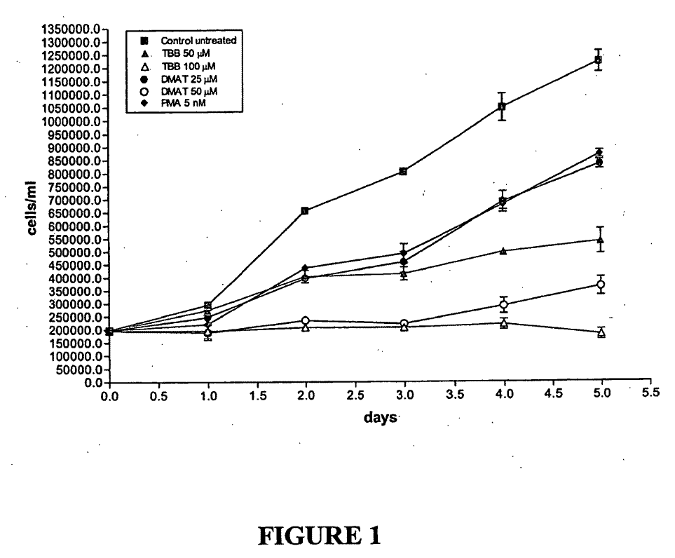

The effect of CK2α inhibitors (TBB and DMAT) on the proliferation rate of MEG-01 cells versus untreated MEG-01 cells (the control) using Trypan blue proliferation assay was investigated as follows. Due to the fact that DMAT and TBB were solubilized in DMSO, a mock control (DMSO only) was used versus untreated MEG-01 cells. This shows that DMSO has no effect on MEG-01 cells regarding proliferation rate and apoptosis level (data not shown). Referring to FIG. 1, the proliferation assay with Trypan blue exclusion is illustrated. From FIG. 1 it can be seen that high concentrations of CK2 inhibitors, such as DMAT 50 μM and TBB 100 μM, maintained a steady number of cells for the 4 days of treatment and induced extremely significant reduction in the proliferation rate of MEG-01 cells. Lower concentrations of inhibitors have the same effect, but less potent during a longer period of time such as 4 days. Cells were maintained basically at about the same number for 4 days, without any decrease from this point suggesting that such inhibitors are capable of regressing the malignant proliferation in MEG-01 cells, without killing them. In contrast, in the absence of inhibition there is a 6-fold increase in cell number.

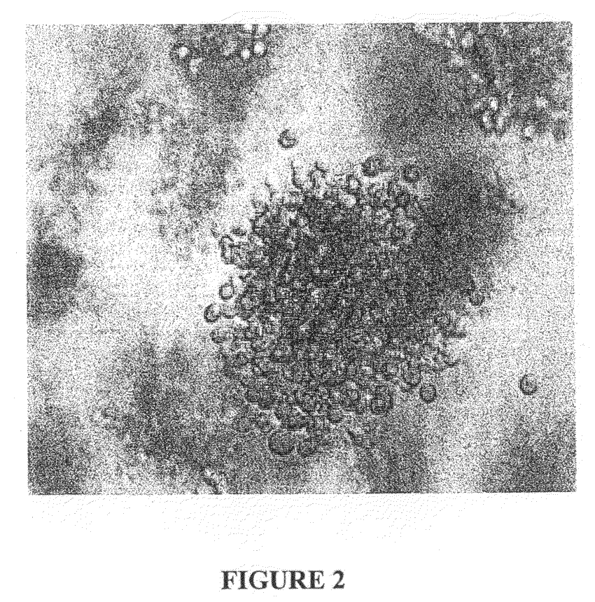

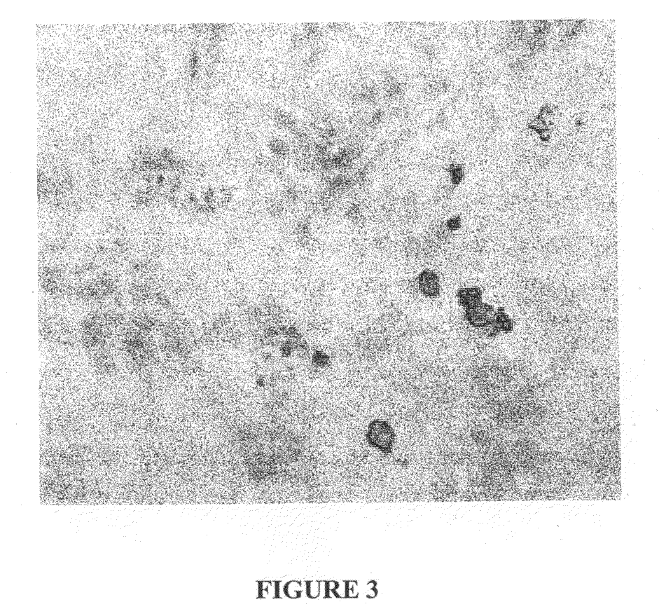

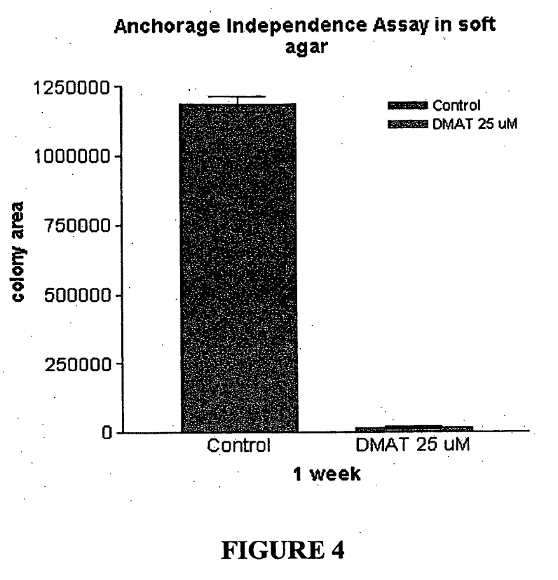

The effect of DMAT treatment on the malignant potential of MEG-01 cells, using anchorage independence assay in soft agar was also investigated as follows. This assay shows that CK2α inhibition by DMAT reduces the malignant potential of MEG-01 cells completely, as shown in FIGS. 2-4. The area and the number of the colonies formed by untreated MEG-01 cells are relatively large (FIG. 2), whereas the treated cells do not form colonies at all (FIG. 3). These results are summarized as measured colony areas, in FIG. 4, for 85 colonies from 30 images.

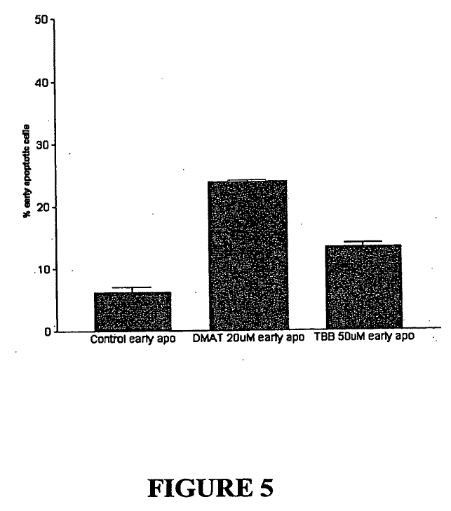

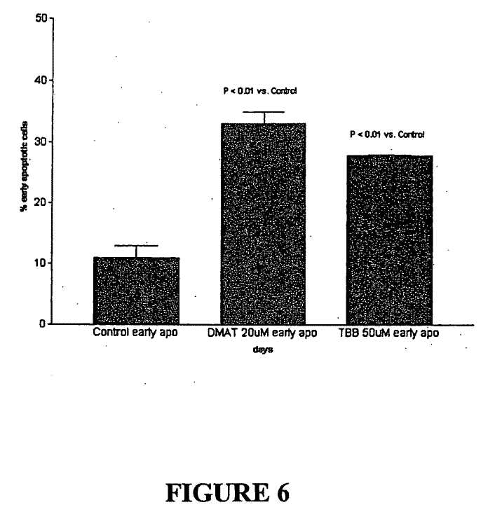

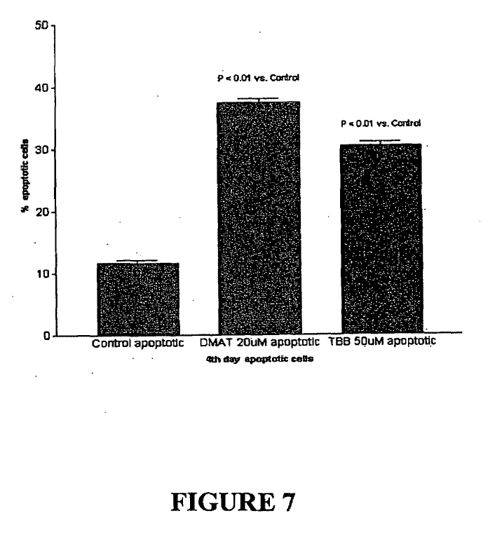

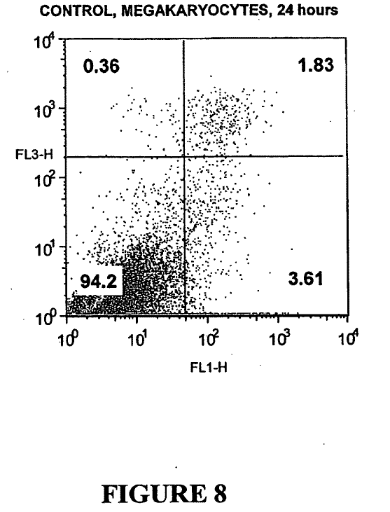

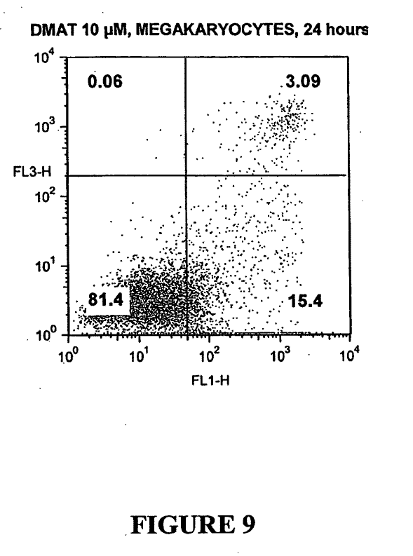

Next, in order to determine if the treatments are not cytotoxic, the apoptosis and dead levels were assessed, using an apoptosis assay with Annexin V-FITC and propidium iodide (PI). FIGS. 5-9 illustrate the results from apoptosis assay with Annexin V-FITC and PI. In FIG. 5, which shows percent apoptotic cells after 24 hours, DMAT 20 μM induces extremely significant apoptosis versus the non-treated control (p<0.01) and TBB 50 μM induced significant apoptosis (p<0.05), similar with DMAT 10 μM induced apoptosis (FIG. 9). After 72 and 96 hours of treatment (see FIG. 6 and FIG. 7, respectively), all the treatments induced extremely significant apoptosis (p<0.01). FIG. 8 shows the dot plot and quadrant gating for control untreated (DMSO) stained for both PI and AnnexinV-FITC after 24 hours. FIG. 9 illustrates the dot plot and quadrant gating for DMAT 10 μM treatment stained for both PI and AnnexinV-FITC and PI after 24 hours. Apoptosis assay shows percent dead cells similar with the control (corresponding to necrotic cells gate, FL1H positive, FL3H positive).

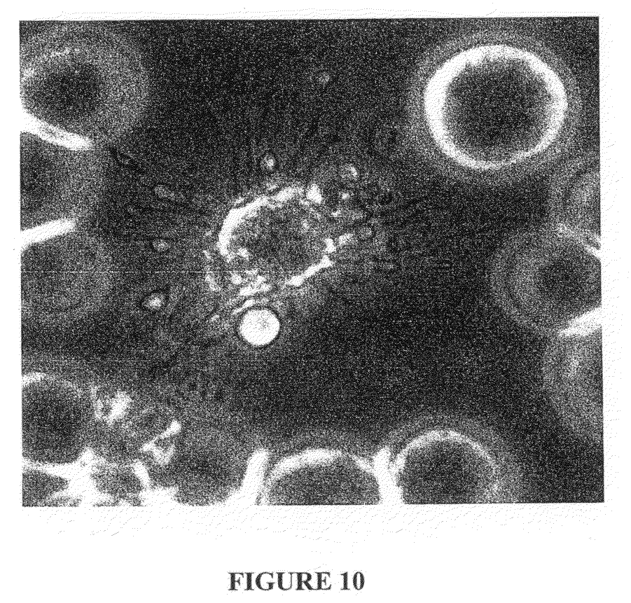

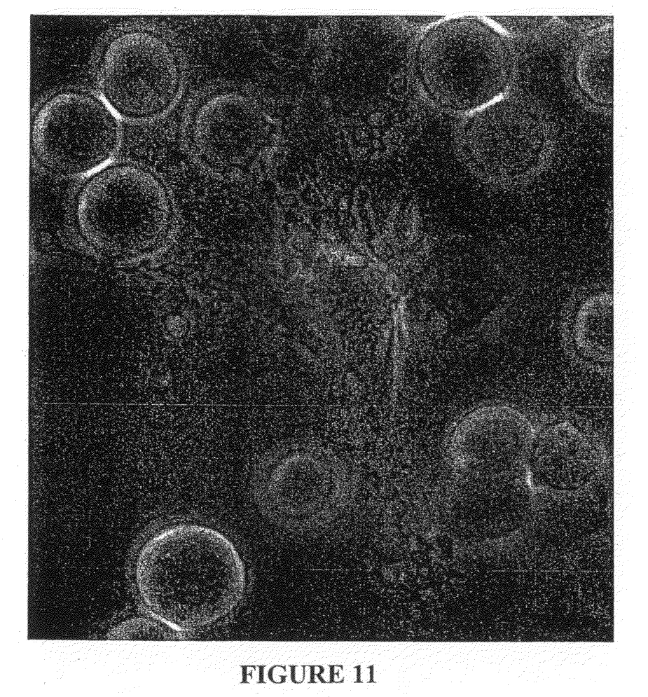

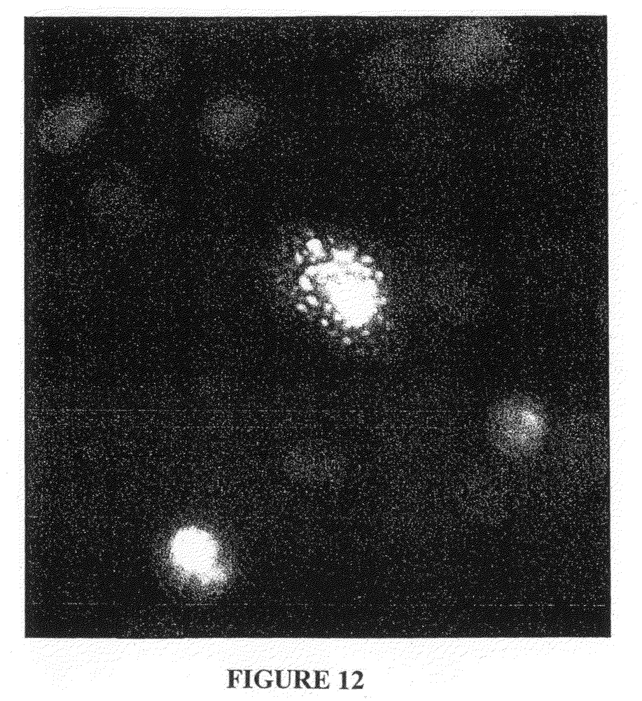

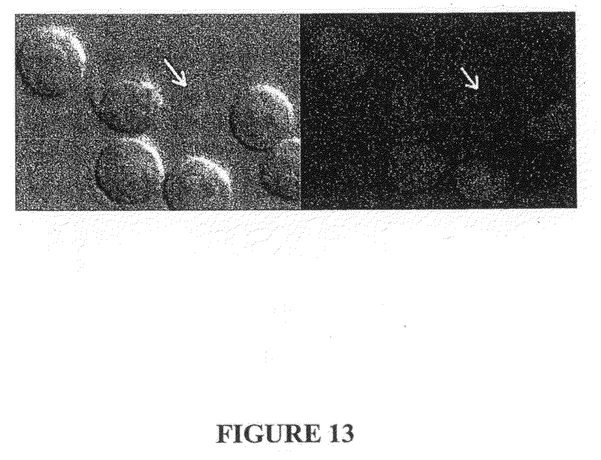

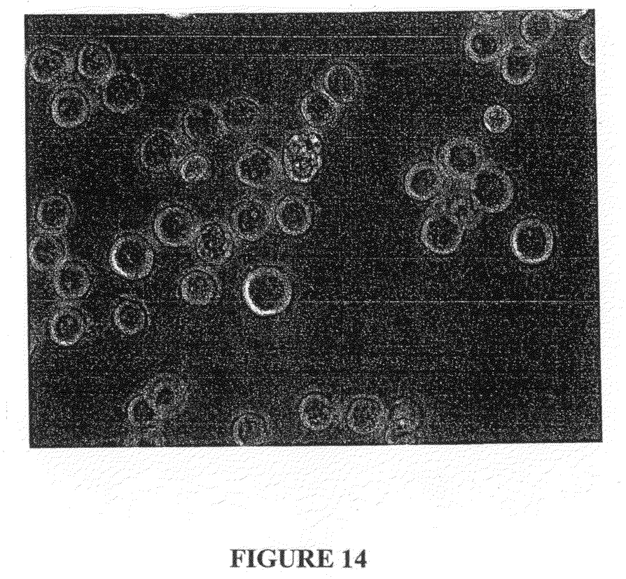

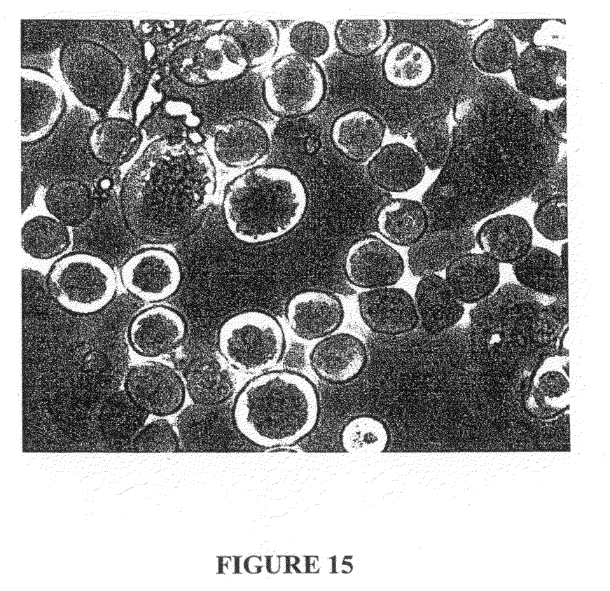

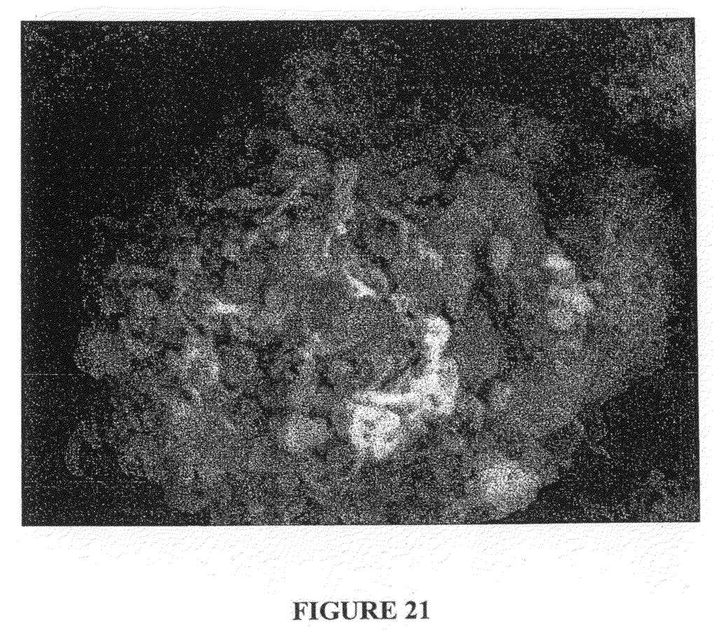

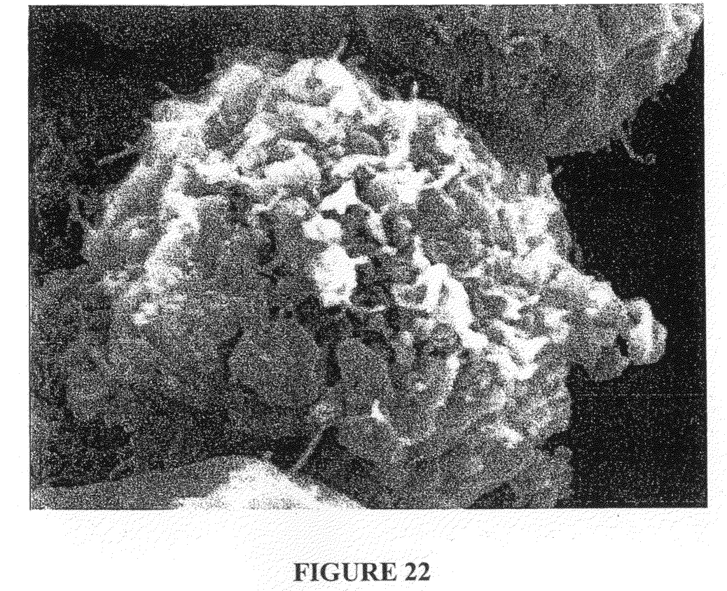

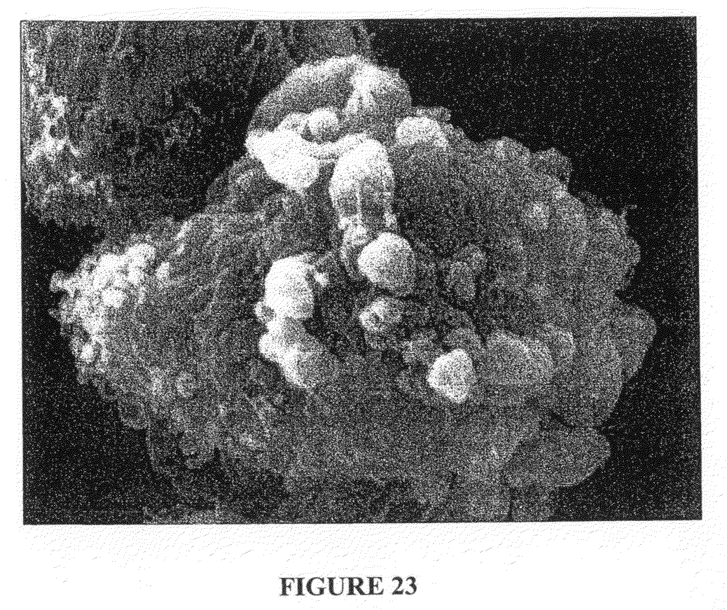

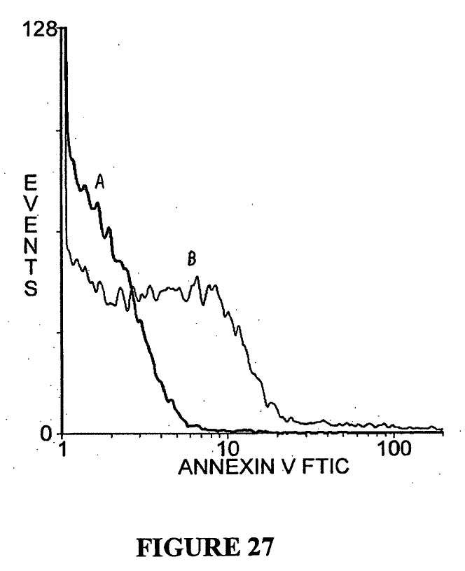

Due to the fact that DMAT and TBB induced apoptosis in MEG-01 cells (and other apoptosis inducers were proven to induce platelets release from MEG-01 cells), an investigation was undertaken to determine if CK2α inhibitors could also be thrombocytopoiesis inducers. It was determined that CK2α inhibition with DMAT and TBB induced thrombocytopoiesis. As early as from the second day, DMAT 10 μM induced MEG-01 cells to mature and form proplatelets extensions, as seen in FIGS. 10 and 11. This process was significantly enhanced in the third and fourth day. The megakaryocytes undergoing thrombocytopoiesis showed apoptotic features, DNA condensation and fragmentation (FIG. 12), blebbing (FIGS. 21-23) and phosphatidylserine exposure (FIG. 27). MEG-01 cells increase in size and become polyploid, as observed by light microscopy micrographs and DAPI staining (FIGS. 10, 11, and 12). Following explosive fragmentation, long filaments with beaded ends (proplatelets) are formed (FIG. 10 in suspension and FIG. 11 on fibronectin). The proplatelets do not stain positive in DAPI (FIG. 12 versus FIG. 11), showing that the beaded ends indeed will become platelets (which are anucleated cells, FIG. 13). Platelets are expelled out from the proplatelets, and the fragmented nucleus slowly is extruded. In FIG. 15 a phase contrast micrograph of DMAT 10 μM on fibronectin is shown in the third to forth day of treatment. This micrograph shows a different phenotype (blebbing, proplatelet formation, increase in size, adherent cells) versus FIG. 14, Control untreated (DMSO) on fibronectin.

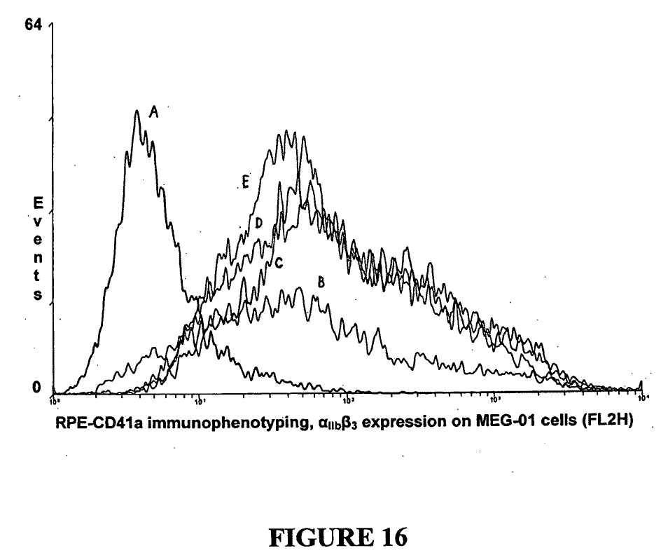

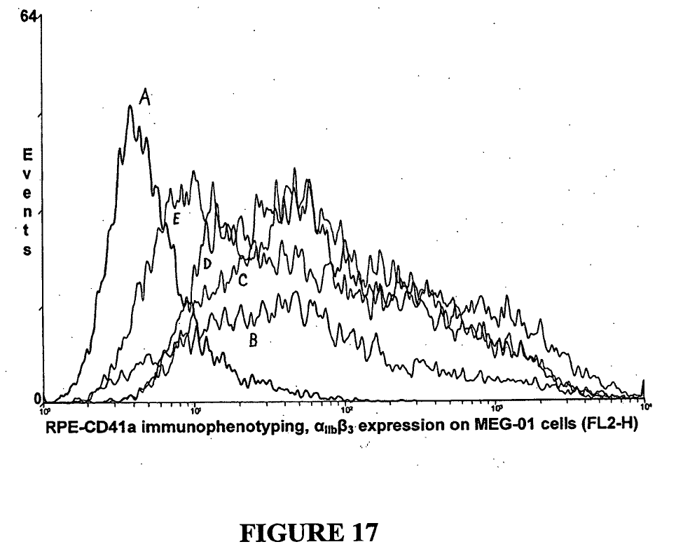

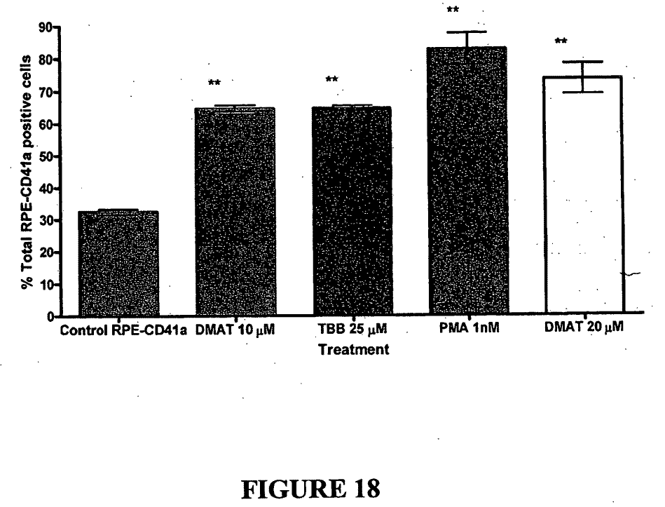

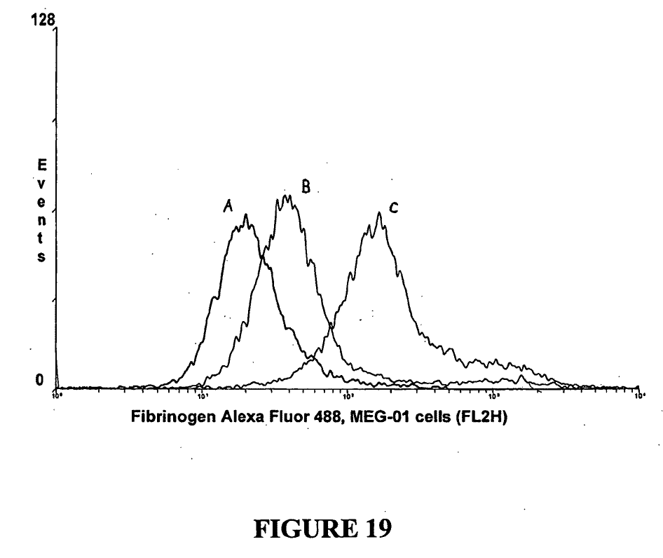

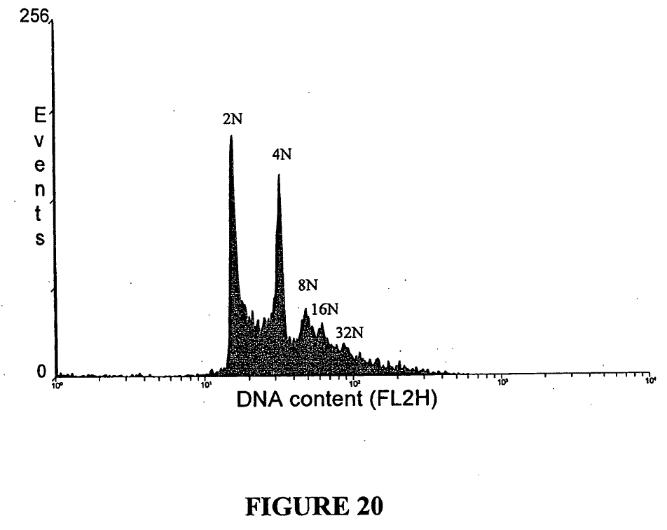

The thrombocytopoiesis process follows the maturation and differentiation process of MEG-01 megakaryoblasts. This maturation process was assessed by the means of flow cytometric immunophenotyping, considering the αIIbβ3 integrin maturation marker. αIIbβ3 integrin increase in expression is correlated with increase in size and differentiation of megakaryoblasts, as seen in FIGS. 16-20. FIG. 16 shows how the DMAT treatment affects maturation level of MEG-01 cells. This Figure shows that as little as DMAT 10 μM concentration (plot D) is sufficient to obtain very significant maturation levels versus control untreated. An increase in the concentration of DMAT up to 20 μM (plot E) induces a slight increase in the maturation level of MEG-01 cells treated. It is also evident that DMAT 20 μM (plot E in FIG. 16) induces similar maturation levels as 1 nM PMA (plot E in FIG. 17) and TBB 25 μM (plot D, FIG. 17). FIG. 18 summarizes the results shown for one set of experiments in FIGS. 16 and 17, using repeated measurements (triplicates). These results are correlated with the proliferation and apoptosis assays. FIG. 19 shows fibrinogen binding to MEG-01 cells, as another maturation marker that shows increase in the fibrinogen receptor expression. In the FIG. 19, MEG-01 cells bind significantly better fibrinogen-Alexa Fluor 488 (plot C) than the untreated controls (plots A and B). DNA content assay with PI and RNAase A shows that MEG-01 cells become polyploid (ploidy higher than 2N) due to DMAT 10 μM treatment, see FIG. 20. Increase in DNA content and cell size, as seen with light microscopy technique (FIGS. 10-15) demonstrates that MEG-01 cells indeed mature due to CK2α inhibitors treatments.

FIGS. 21-23 show SEM microscopy results for MEG-01 cells treated with DMAT 10 μM for 3 to 4 days. Pseudopodia and proplatelets formation, as well as blebbing can be observed at the surface of these cells.

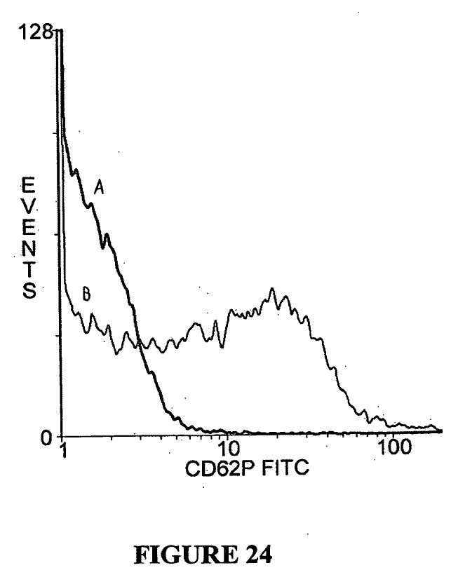

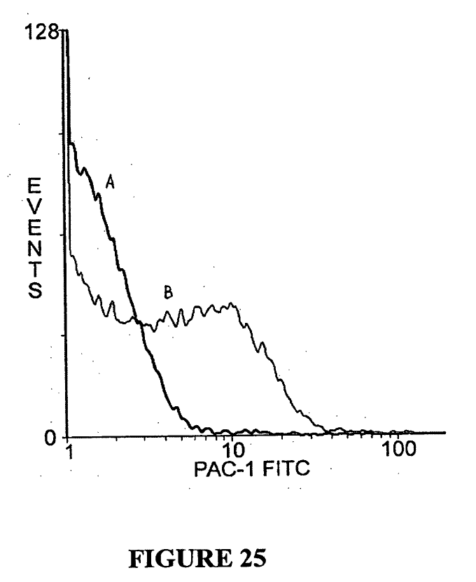

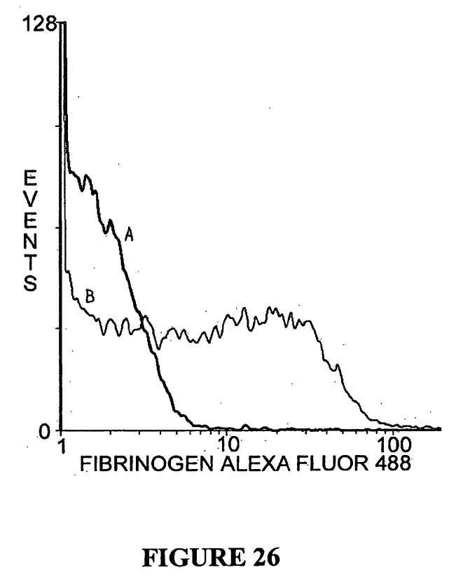

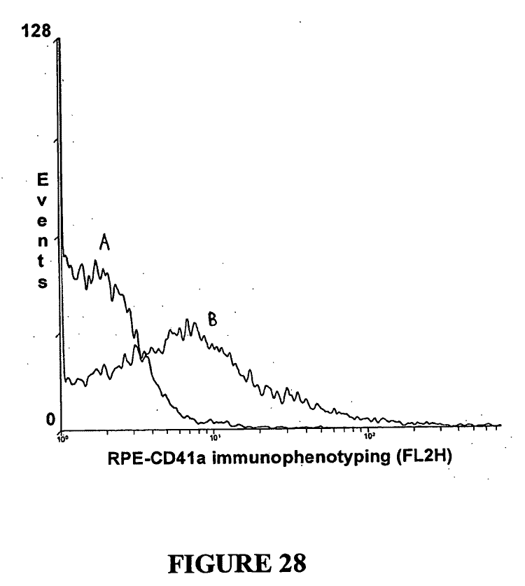

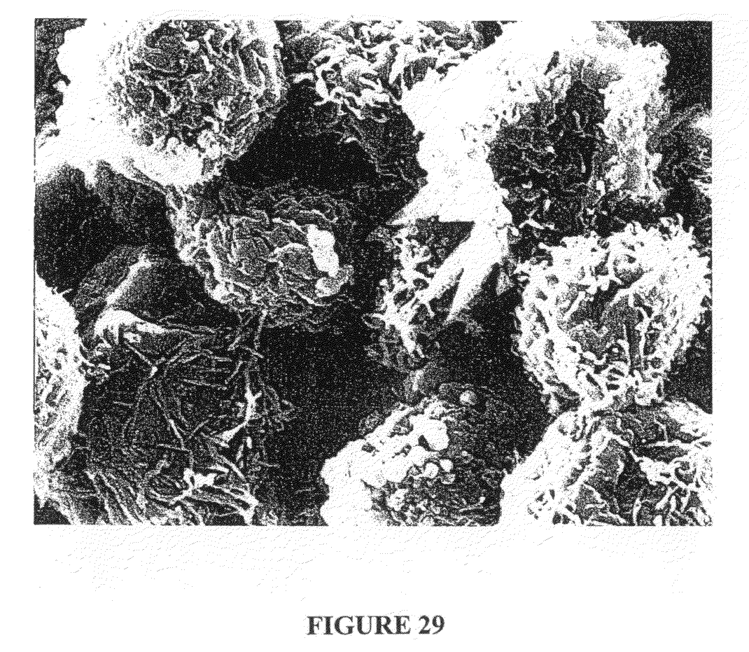

It has been demonstrated that in accordance with the present invention, CK2α inhibition results in apoptosis, proliferation arrest, maturation of MEG-01 cells that concludes with thrombocytopoiesis. Therefore, analysis was conducted to determine whether the platelets produced due to these treatments were functional and that CK2α inhibition with DMAT in the progenitor cell line does not affect the progeny. After functional studies, it was concluded that the platelets obtained in culture, following CK2α inhibition with specific kinase inhibitors (DMAT and TBB), are functional, as shown in FIGS. 21-23. Such platelets were identified as being anucleated (DAPI staining with fluorescence microscopy, PI staining with flow cytometry) and have the size of 1-4 μm, see FIG. 13 and FIGS. 24-28. The platelets are capable of undergoing shape change in response to agonists (like human thrombin, TRAP, ADP, PMA). These platelets form a clot visible with the eye when activated with 0.5 U/ml human thrombin, for which we did scanning electron microscopy (SEM), as seen in FIGS. 29-31. Note the platelets spiked appearance, how they aggregate to each other and how the fibrin net connects them. Such activated platelets stain positive for PAC-1 (an antibody that recognizes a specific epitope on αIIbβ3 integrin, exposed only when platelets are activated), FIG. 25. These platelets expose P-Selectin (FIG. 24) and phosphatidylserine (FIG. 27) and bind fibrinogen (FIG. 26), as well as CD41a (an antibody that recognizes αIIbβ3 integrin on both activated and inactivated platelets), FIG. 28.

As used herein, the term “effective amount” means a dosage sufficient to produce a desired or stated effect.

As used herein, the term “leukemia” generally refers to cancers that are characterized by an uncontrolled increase in the number of at least one leukocyte and/or leukocyte precursor in the blood and/or bone marrow. Leukemias including but not limited to acute lymphoblastic leukemia (ALL); acute myeloid leukemia (AML); chronic lymphocytic leukemia (CLL); chronic myelogenous leukemia (CML); and, hairy cell leukemia are contemplated. “Leukemic cells” typically comprise cells of the aforementioned leukemias.

The methods of the invention may be applied to cell populations in vivo or ex vivo. “In vivo” means within a living individual, as within an animal or human. In this context, the methods of the invention may be used therapeutically in an individual, as described herein.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. Information gleaned from such use may be used for experimental purposes or in the clinic to set protocols for in vivo treatment. Other ex vivo uses for which the invention may be suited are described below or will become apparent to those skilled in the art. Ex vivo applications include in vitro applications, studies, and investigations.

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

“Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma et al. (Eds.), Goodman and Gilman's The Pharmacological Basis of Therapeutics, 9th ed., pp. 11-16 (1996). A thorough discussion is provided in Higuchi et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

The present invention further provides kits for disease diagnosis, prognosis, risk assessment, and/or treatment efficacy determination. Such kits are useful in a clinical setting for use in diagnosing a patient for a disease, monitoring the disease progression, testing patient's samples (e.g. biopsied), for example, to determine or predict if the patient's disease (e.g., cancer) will be resistant or sensitive to a given treatment or therapy with a drug, compound, chemotherapy agent, or biological treatment agent.

### Procedures

In accordance with the present invention, the effect of the inhibition of CK2α with specific kinase inhibitors, TBB and DMAT on a malignant megakaryoblastic cell line MEG-01 was considered. This work was undertaken to better understand the effect of CK2 on the thrombocytopoiesis process.

### Cell Culture

MEG-01 megakaryoblastic cell line (Ogura et al., 1985), was a generous gift from the Department of Biochemistry, University of Vermont, College of Medicine, Burlington, Vt., USA. Cells were grown conforming with directions from American Type Culture Collection (Manassas, Va., USA). Shortly, cells were maintained in a VWR incubator, with a humidified atmosphere of CO₂ 5%, and at 37° C. Cells were usually cultured in plastic tissue culture flasks with green quick-release screw cap, vol. 50 ml, 25 cm² growth area, with ventilation, from Sarstedt, Newton, N.C., USA. Cell culture media RPMI 1640 1× with L-Glutamine (2 mM) from Central Cell Services, Media Lab, Lerner Research Institute, Cleveland Clinic Foundation, Cleveland, USA, was adjusted to contain 10 mM HEPES (Invitrogen, Carlsbad, Calif., USA), and 1.0 mM Sodium Pyruvate (Invitrogen, Carlsbad, Calif., USA), 90% and 10% heat-inactivated fetal bovine serum (Invitrogen, Carlsbad, Calif., USA) and Penicillin/Streptomycin 100 U/ml, 100 μg/ml Invitrogen, Carlsbad, Calif., USA. Cells were seeded at 1-2×105 cells/ml, media was renewed and cell number adjusted two times per week. Some of the cells become adherent as they mature, therefore the culture is presented as a population of cells in suspension and one slightly adherent to the flask. Repeated washings with the culture media gently collected adherent cells. The total removal of the adherent cells from the culture dish was checked at the microscope each time.

For observing the cells with light microscopy and to be able to provide a matrix for them to adhere, Fibronectin (FN), was used at 5 μg/cm² and incubated at 37° C. for 1 h, as a coat for the culture dish (6 well plates usually), as recommended by the provider (BD Biosciences, Bedford, Mass., USA) (Jiang et al., 2002).

For viability assay, flow cytometry and platelets assays cells were grown without the FN coated substratum, because FN adheres to cells also via its RGDS sequence.

HUVEC cells were grown on Fibronectin coat (at 5 μg/cm²) until they reached 70% confluence, at which were split after trypsinization with Trypsin/EDTA (Invitrogen). DMEM 1× (from Central Cell Services, Media Lab, Lerner Research Institute, CCF) supplemented with FCS10%, Penicillin/Streptomycin 100 U/ml and 100 μg/ml, L-Glutamine (2 mM) and endothelial cell growth factor ECGF from bovine brain extract (100 μg/ml) and 50 μg/ml heparin, was used as a growth media.

### Cell Treatments

Cells were treated with the CK2α inhibitors, 4,5,6,7-Tetrabromobenzotriazole (TBB), and 2-Dimethylamino-4,5,6,7-tetrabromo-1H-benzimidazole (DMAT) (Pagano et al., 2004b; Ruzzene et al., 2002). The vehicle for DMAT, TBB is DMSO. As a differentiation and apoptosis control, besides the untreated control (DMSO only), Phorbol 12-myristate 13-acetate (PMA) was used (Baatout, 1998). PMA is a very potent tumor promoter and platelet activator, which induces differentiation in different cell lines, by activating Protein Kinase C (PKC) groups.

TBB, DMAT, PMA and DMSO were purchased from Calbiochem (EMD Biosciences), San Diego, Calif., USA.

In order to decide the best treatment concentrations to use, titrations of inhibitors were done. Apoptosis and viability assays were run to choose the non-cytotoxic and statistical significant concentrations of TBB and DMAT that have significant effect.

At the start of the treatments the cells were counted with the hemacytometer and splitting was done in order to have for each sample, the same cell number of 2×105 cells/ml. In order to check the effect of CK2 inhibitors, the treatments length went up to 4, 5 days without splitting.

### Proteins, Peptides and Fluorochromes, ADP Used in Platelets and Apoptosis Assays

Human fibrinogen, ADP, TRAP were kindly provided by the Cleveland Clinic Foundation, Cleveland, Ohio, USA. CD62P (anti human P-Selectin monoclonal antibody) conjugated with FITC, CD41a (anti human αIIbβ3 monoclonal antibody) conjugated with RPE, PAC-1-FITC antibody and AnnexinV conjugated with FITC and the PI (AnnexinV-FITC and PI Apoptosis Kit I), were all purchased from BD Biosciences, Bedford, Mass., USA. The fluorochrome Alexa Fluor 488 was purchased from Molecular Probes, OR, USA.

### Apoptosis Assays Using Flow Cytometry with AnnexinV-FITC and PI

For induction of typical apoptosis, cells were cultured with 1 μM Staurosporine (Sigma-Aldrich, cat. # S6942) for 6 hours as recommended in the protocol from the provider. Next the Staurosporine treated cells were stained as follows: (1) with PI, (2) with AnnexinV-FITC and (3) both PI and AnnexinV-FITC in order to have the brightest controls for compensation and proper collection of the flow cytometry data. Cell necrosis was induced by heat shock (65° C. for 30 minutes). Controls for necrosis were stained in the same way as for the Staurosporine treated cells.

In order to establish the effect of the treatments on the apoptosis levels, the non-treated cells (as well as the mock control DMSO) were considered as a control, stained with PI and AnnexinV-FITC and the unstained cells, grown for the same length of treatment. The unstained control signal was subtracted by gating from the stained control and the rest of stained samples, to get only the positive stained cells signal. The stained control, both PI and AnnexinV-FITC labeled, was then used as the reference in establishing the level of apoptosis induced by the treatments.

Samples contained three cell populations of different size and shape and data was collected on logarithmic modes. AnnexinV-FITC corresponds to FL1H channel, and PI to FL3H or FL2H channels. When needed, compensation was done to remove any spillover between the channels. In addition, proper gating was done to characterize each cell population. The population of cells high on FSC and SSC, identified as large, non-granulated particles corresponds to MEG-01 cells. This population is involved in the proplatelets formation and terminal phase of megakaryocytopoiesis and in thrombocytopoiesis. MEG-01 gate is the one of interest in estimating the apoptosis level induced by the treatments.

The population that corresponds to platelets, is PI negative because thrombocytes are anucleated cells (only the viable cells were considered) and were distinguished by their capacity to get activated, undergoing shape change and moving up on SSC as response to agonist and to show phosphatidylserine (PS) exposure when activated (Clarke et al., 2003).

Platelets were also separated from the megakaryocytic cells, by differential centrifugation (Zunino et al., 2001), considering the big size difference between these cells (1-5 μm for platelets and 35-150 μm for megakaryocytic cell line MEG-01) and analyzed separately. Voltage and channels settings were adjusted accordingly. Cells were collected using a FACSCalibur machine with CellQuestPro version 3.3 software and analyzed using Mac OS X version of FlowJo version 6.2., and the Windows version of WinMDI 2.8.

### AnnexinV-FITC and PI Apoptosis Assay Staining Protocol

The Apoptosis AnnexinV-FITC and PI kit from BD Biosciences, CA, USA was used. 105-106 cells were stained with 50 μg/ml propidium iodide (PI) and with 0.5 μg/ml FITC-labeled Annexin V using the staining protocol provided by the supplier. Briefly, cells were counted using a Neubauer hemacytometer, then washed and resuspended in the appropriate amount of AnnexinV-binding buffer 1× (provided in the kit), in order to have the most 106 cells in 100 μl. Then it was used further, as the total reaction volume 100 μl (containing 106 cells) to which PI and AnnexinV-FITC was added. Incubation was done for 15 minutes in dark at room temperature. Samples were then analyzed immediately by flow cytometry.

### RPE-CD41a and Alexa Fluor 488 Fibrinogen, Immunophenotyping of MEG-01 Cells

CD41a is the antigen for αIIbβ3 complex and it is found on platelets and platelet precursors, including MEG-1 cell line (Ogura et al., 1985; Zunino et al., 2001). It acts as a receptor for fibrinogen, von Willebrand factor, fibronectin, and vitronectin, and it mediates platelet adhesion and aggregation. αIIbβ3 complex is a marker of differentiation for megakaryocytes (as an increase in the fluorescence of stained cells corresponds to more αIIbβ3 receptor expression and differentiation towards platelets production) and it is simply a marker for the platelets. CD41a is conjugated with R-Phytocoerthrin (RPE), in order to be used in flow cytometry experiments. The staining protocol provided by BD Biosciences was followed. Briefly, cells were washed and resuspended in 1×PBS with 0.1% FBS, 0.01% NaN₃, and 0.22 μm filtered buffer. Cells were counted and adjusted to be 106 cells/ml and 20 μl of RPE-CD41a was used for 180 μl cell suspension.

RPE conjugated stained cells were collected on FL2H channel and gating on populations was done on FSC and SSC logarithmic modes. The unstained control signal was subtracted from the stained cells signal, in order to measure the staining of the cells without background noise. Similar staining was done for Fibrinogen-Alexa Fluor 488 (300 nM). Cells were collected on FL1H channel and gating on the interest population was done on FSC and SSC logarithmic modes. Again, the unstained control signal was subtracted from the stained cells signal, in order to measure the staining of the cells without background noise.

### Platelets Activation and Function Assessment by Flow Cytometry Platelets Isolation from Culture

Platelets were also separated from the megakaryocytic cells, by differential centrifugation (Zunino et al., 2001), considering the big size difference between these cells (1-5 μm for platelets and 35-150 μm for megakaryocytic cell line MEG-01) and analyzed separately. Suspension cells were centrifuged first at 100 g to 150 g for 15 minutes, and then the platelets-rich supernatant was kept and centrifuged again at 800 g for 15 minutes. The platelets pellet was resuspended in the appropriate buffer (Tyrode's buffer, pH 7.4), for analysis. For impeding artefactual aggregation of platelets in the control, EDTA and RGDS or RGD were added in the cell suspension from the beginning of the centrifugations. Therefore the control was the inactivated platelets. Special caution was used in order to avoid as much as possible artefactual activation of the control, therefore centrifugations were done at room temperature, no unnecessary washing, no vortexing, gentle removal of supernatants.

### PAC-1-FITC Binding Due to Platelet Activation Flow Cytometric Assay

Monoclonal antibody PAC-1 recognizes an epitope on the glycoprotein αIIbβ3 of activated platelets. PAC-1 binds only to the activated platelets, and so is very specific. PAC-1 will not bind EDTA and RGD or RGDS treated platelets (Shattil et al., 1987; Shattil et al., 1985; Shattil and Leavitt, 2001).

10 mg/ml RGD or RGDS and 100 mM EDTA were used for negative control (inactivated) on resting platelets.

PAC-1 is conjugated with the FITC fluorochrome for flow cytometry experiments (488 nm laser excitation, FL1H channel).

PAC-1-FITC was purchased from Becton Dickinson Immunocytometry Systems, San Jose, Calif., USA. 20 μl PAC-1-FITC were used for 5 μl fresh platelets suspension, in Tyrode's buffer with CaCl₂, as recommended for whole blood by the provider. Different agonists were used, like 1 μg/ml TRAP, 100 nM PMA, as final concentrations (Chen et al., 2004). Activation time was 10-15 minutes.

BD Biosciences protocol for staining was used, with incubation for 30 minutes, in dark, at room temperature. The assays were done on fresh platelets, not on fixed ones, because fixed platelets do not bind PAC-1.

### CD62P-FITC Exposure Due to Platelet Activation Flow Cytometric Assay

CD62P is a monoclonal antibody that recognizes an epitope on P-Selectin. P-Selectin is exposed as response to agonist and is a specific sign of platelet activation. 20 μl CD62P-FITC were used for 5 μl fresh platelets suspension, in Tyrode's buffer with CaCl₂. Different agonists were used, like 1 μg/ml TRAP, 100 nM PMA or 20 μM ADP as final concentrations. Activation time was 10 minutes. The staining volume was chosen by titration, small volumes giving better staining. Incubation was done in dark at room temperature for 30 minutes, as recommended by BD Biosciences. These assays were done on fresh platelets.

### Fibrinogen-Alexa Fluor 488 Binding to Platelets Flow Cytometric Assay

Human fibrinogen was conjugated with Alexa Fluor 488 fluorochrome (F-13191, from Molecular Probes, OR, USA) following recommended Molecular Probes procedure, with the modification that instead the column step, a dialysis step was chosen, in order to obtain a better attachment of dye molecules to fibrinogen. Final concentration of fibrinogen conjugated Alexa Fluor 488 was determined spectrophotometrically. For platelet activation, different agonists were used, like 1 μg/ml TRAP, 100 nM PMA or 20 μM ADP as final concentrations. Activation time was 10-15 minutes. 300 nM labeled fibrinogen was incubated as a final concentration with platelets suspension for 30 minutes, in dark, at room temperature. Fresh platelets were used.

### AnnexinV-FITC for Phosphatidylserine (PS) Exposure

Platelets expose PS when activated and undergo shape change. They will stain positive for AnnexinV (a marker of PS exposure) and will move up on the SSC, FSC as a result of the shape change. For this assay, AnnexinV-FITC from BDBiosciences was used, as recommended by the manufacturer with the specification that the reaction volumes were decreased. Incubation was done in dark, at room temperature for 30 minutes, after activation for 10 minutes of platelets with agonist (like 1 μg/ml TRAP, 100 nM PMA or 20 μM ADP as final concentrations).

### Viability (Proliferation) Assay, Trypan Blue Exclusion

Cells were counted using a Neubauer hemacytometer. Trypan blue dye was used according to the manufacturer (Sigma-Aldrich, cat. # T8154).

Cell number (as cells/ml) was estimated using the formula [(total count/0.1)/total surface area counted]*(dilution factor), where 0.1 is the chamber depth, considering the appropriate area counted, half dilution for Trypan blue was considered in calculations. Countings were done as quadruplicates every 24 hours. DMSO, which is the vehicle for TBB, DMAT and PMA, was used as a mock control to compare with the untreated control, considering the highest amount that was used as a vehicle for TBB and DMAT. DMSO was proven by both apoptosis and proliferation assay to have no statistically significant effect on the MEG-01 cells.

### Anchorage Independence in “Soft Agar” Assay

Anchorage independence of growth in soft agar assay is an assay for detecting malignant transformation of cells. Anchorage independence is given by defects in the cellular adhesion pathways. Anchorage independence is strongly connected with tumorogenicity and invasiveness. Malignant cells form colonies when grown on soft agar. Normal cells cannot grow on soft agar. Agarose (V3121 from Promega, with melting point at 87-89° C. and gelling point at 36-39° C.) was mixed with MEG-01 growth media RPMI1640 1× with 10% FBS and supplements. A bottom layer of 0.5% makes a firm base, then a top layer of 0.3% agarose with media and over the agarose a layer of growth media was added. Cells were grown in 12 wells dishes at 37° C., 5% CO₂, 90% humidity in a VWR incubator. Colonies formation was observed and photographed using an Olympus CK40 microscope, in the laboratory of Cleveland State University, Biology Department. Cells were observed after one week. One control population was used and one treated with DMAT 25 μM. Media was renewed on the top of the agar every 4 days. Analysis of the pictures and measurements of the colonies areas were done using the NIH Image version 1.63 for MacOS 9 microscopy software. Good quality pictures were transformed into binary gray images and then white background was removed using a 50 ball radius. This modified image was then transformed into a default threshold image and then colonies were gated and measured.

### Light Microscopy (Phase Contrast) and DAPI Fluorescence Microscopy

Cell pictures were routinely taken in the laboratory of using an Olympus CK40 microscope, and the NIH Image software. High quality pictures and live imaging were taken at Cleveland Clinic Foundation Imaging Core.

DAPI staining of cultured MEG-01 cells was done with fresh cells, for live imaging.

Different concentrations of inhibitors were used, and both floating as well as fibronectin bound cells were examined. Proplatelets producing cells were observed as early as from the second day (after 24 hours), but in the third and forth days proplatelets formation was enhanced. From the third to the forth day, cells were observed for a period of 24 hours in order to catch cells undergoing thrombocytopolesis. When such a cell was identified, efforts were taken to focus on it and pictures were taken every 10 seconds for one day.

### Scanning Electron Microscopy (SEM)

Cell preparation and pictures were done at the Microscopy Core Facility Cleveland Clinic. Cells were grown for 4 days with DMAT 10 μM and then collected by differential centrifugation. Cells were fixed in glutaraldehyde buffer and then further processed at the Core. The clot was prepared with platelets collected from MEG-01 cultures treated with DMAT 10 μM. Human thrombin 0.5 U/ml for 15 minutes was used to activate them. Then the clot was fixed in suspension.

More specifically, referring to FIG. 1, cell proliferation (viability) assay, with Trypan Blue exclusion is illustrated. Five days of treatment, proliferation (viability) of MEG-01 cells. Filled square represents Control untreated, filled triangle is TBB 50 μM, open triangle is TBB 100 μM, filled circle is DMAT 25 μM, open circle is DMAT 50 μM and filled diamond is PMA 5 nM. Statistical analysis and graphing was performed using GraphPad, Prism software version 2.01. For each sample, Column Statistics was applied (samples passed Normality test and between multiple readings for each sample p>0.10 shows no statistical differences between results obtained in different experiments for the same concentration and length of treatment, therefore readings were considered accurate). One-Way ANOVA Test-Repeated Measures followed by Dunnett's Multiple Comparison Test (which compares all treatment columns vs. the control column) shows a p<0.05 * for TBB 100 μM and DMAT 50 μM vs. Control untreated (DMSO), from the first day. In the second day (after 24 hours), p<0.05 * is seen for all the treatments (TBB 50 μM, TBB 100 μM, DMAT 25 μM, DMAT 50 μM) versus the Control untreated (DMSO). In the fourth and fifth days values of p<0.01 ** for TBB 50 μM and DMAT 25 μM and p<0.005 for TBB 100 μM and DMAT 50 μM show very significant statistical differences. Quadruplicates values were used for each sample. Experiments were repeated at least 4 times, in order to see if the trend is correct and reproducible.

Referring to FIGS. 2-4, anchorage independence assay in soft agar (0.6% base and 0.3% top layer agar, with complete MEG-01 cells, growth media) is illustrated. Malignant cells form colonies when grown on soft agar. The area of each colony was measured using the software NIH Image version 1.63. First the background was subtracted from a gray binary image, then default threshold was applied and then colonies were measured by appropriate gating. 85 colonies from 30 images were measured. Cells pictures and analysis was done using an Olympus CK40 microscope, using a 20× magnification. In FIG. 2, control untreated colony formation is illustrated in soft agar by MEG-01 cells. FIG. 3 illustrates DMAT treated (25 μM) colony formation in soft agar by MEG-01 cells. FIG. 4 illustrates comparison of colony sizes between Control untreated and DMAT treated MEG-01 cells. Control untreated left column, DMAT treatment right column. Error bars are SD, p<0.0001 (extremely significant) with paired student t-test (GraphPad Prism).

With reference to FIGS. 5-9, apoptosis flow cytometry (AnnexinV-FITC corresponding to FL1H and PI corresponding to FL3H) is depicted. Analyzed and graphed values correspond to frequencies of the parent obtained with FlowJo software version 6.2, after quadrant gating. P value was determined with One-Way ANOVA Test-Repeated Measures followed by Dunnett's Multiple Comparison Test (which compares all treatment columns versus the control column). In FIG. 5, apoptotic cells, percentages (FL1H+, FL3H−), after 24 hours of treatment with CK2α inhibitors are depicted. Left bar represents Control untreated (DMSO), middle bar represents DMAT 20 μM treatment and right bar represents TBB 50 μM treatment. The MEG-01 cells were treated for a period of 24 hours. Apoptosis was measured using flow cytometric assay AnnexinV-FITC and PI. p<0.05 * for treatments versus control untreated (DMSO) shows significant apoptosis after 24 hours. The graph shows percentage of early apoptotic cells (gate FL1H+, FL3H−) as a function of treatment, after 24 hours. FIG. 6 shows early apoptotic cells, percentages (FL1H+, FL3H−), after 72 hours of treatment. Left column represents the Control untreated (DMSO), middle bar represents DMAT 20 μM treatment and right bar represents TBB 50 μM treatment. The MEG-01 cells were treated for a period of 72 hours. Apoptosis was measured using flow cytometric assay AnnexinV-FITC and PI. p<0.01 for treatments versus control untreated (DMSO) shows significant apoptosis after 72 hours and increase in the apoptosis levels with the increase in the treatment length. The graph shows percentage of early apoptotic cells (gate FL1H+, FL3H−) as a function of treatment, after 72 hours. FIG. 7 shows total apoptotic cells (Late apoptotic (FL1H+, FL3H+) plus Early apoptotic (FL1H+, FL3H−)), after 96 hours of treatment. Left bar represents the Control untreated (DMSO), middle bar represents DMAT 20 μM treatment and right bar represents TBB 50 μM treatment. The MEG-01 cells were treated for a period of 96 hours. Apoptosis was measured using flow cytometric assay AnnexinV-FITC and PI. p<0.01 ** for treatments versus control untreated (DMSO) shows significant apoptosis after 96 hours. The most specific inhibitor used (DMAT) induces more apoptosis at a lower concentration than the less specific inhibitor (TBB). The graph shows percentage of total apoptotic cells (gate FL1H+, FL3H−) as a function of treatment, after 96 hours. FIG. 8 illustrates control untreated (DMSO) stained for both Annexin V-FITC and PI, after 24 hours. FIG. 9 shows DMAT 10 μM stained for both AnnexinV-FITC and PI (collection mode FL1H, FL3H) after 24 hours of treatment.

Referring to FIGS. 10-15, DAPI staining of live MEG-01 cells is illustrated. Apoptotic morphology of MEG-01 cells. In FIG. 10, proplatelets formation in suspension, result of DMAT treatment (10 μM) is shown. FIG. 11 depicts proplatelets formation on Fibronectin, result of DMAT treatment (10 μM). FIG. 12 shows proplatelets bearing MEG-01 megakaryocyte, DAPI staining, result of DMAT treatment (10 μM). This image represents the DAPI staining of cells from FIG. 11. FIG. 13 shows platelets identified as anucleated cells with DAPI staining. FIG. 14 shows blebbing of MEG-01 cells treated with 10 μM DMAT. FIG. 14 also illustrates phase contrast micrograph of live MEG-01 cells untreated in the 3rd to the 4th day (DMSO), 20×. FIG. 15 illustrates phase contrast micrograph of live MEG-01 cells treated with DMAT 10 μM in the 3rd to the 4th day, 20×. To summarize, FIGS. 10-15 show chromatin condensation, blebbing, cytoplasmic and nuclear fragmentation, as well as polyploidy (increase in the size of the nucleus) of MEG-01 cells following DMAT treatment.

Regarding FIGS. 16-20, maturation (differentiation) of MEG-01 cells is described. In FIG. 16, RPECD41a (αIIbβ3 expression) staining for DMAT treated cells (gate MEG-01) is depicted, with control untreated unstained—plot A, Control untreated stained (RPE-CD41a)—plot B, DMAT 10 μM—plot D, DMAT 15 μM—plot C, DMAT 20 μM—plot E. For FIG. 17, RPECD41a (αIIbβ3 expression) staining for DMAT, TBB and PMA (gate MEG-01) is shown. Control untreated unstained—plot A, control untreated stained—plot B, 10 μM DMAT—plot C, 25 μM TBB—plot D, 1 nM PMA treatments—plot E. FIG. 18 represents total percentage relative fluorescence for RPE-CD41a immunophenotyping. Control untreated stained (RPE-CD41a), DMAT 10 μM, TBB 25 μM, PMA 1 nM, DMAT 20 μM, are shown in the noted bar graph with p<0.01 between each treatment and untreated values, which conform to One-Way ANOVA, Dunnett's multiple comparison statistical analysis. FIG. 19 shows Fibrinogen-Alexa Fluor 488 staining for DMAT treated MEG-01 cells, Control untreated, unstained—plot A, Control untreated stained—plot B, and 10 μM DMAT—plot C. FIG. 20 shows DNA content of MEG-01 cells treated with DMAT 10 μM for 4 days, shows ploidy higher than 2N as assessed by PI and RNAase A flow cytometric assay.

Referring to FIGS. 21-23, MEG-01 platelet—producing cells phenotype (pseudopodia, proplatelets, blebbing) following DMAT treatment, as observed by SEM is shown.

Referring to FIGS. 24-28, platelets activation and identification by flow cytometry is described. FIG. 24 shows P-Selectin exposure (CD62P—FITC) by platelets when activated by agonist. Control resting—plot A, agonist activated (TRAP)—plot B. These platelets result from DMAT 10 μM treatments. The control was treated with EDTA and RGDS. FIG. 25 shows PAC-1 binding (PAC-1-FITC) by platelets when activated by agonist. Control resting—plot A, agonist activated (TRAP)—plot B. These platelets result from DMAT 10 μM treatments. The control was treated with EDTA and RGDS. FIG. 26 shows Fibrinogen-Alexa Fluor 488 binding to activated platelets. Control resting—plot A, agonist activated (TRAP)—plot B. These platelets result from DMAT 10 μM treatments. The control was treated with EDTA and RGDS. FIG. 27 shows Annexin V binding binding to activated platelets. Control resting—line A, agonist activated (TRAP)—plot B. These platelets come from DMAT 10 μM treatments. The control was treated with EDTA and RGDS. FIG. 28 shows RPE-CD41a staining (marker of platelets), Control untreated unstained, inactivated—plot A, platelets from DMAT 10 μM stained—plot B.

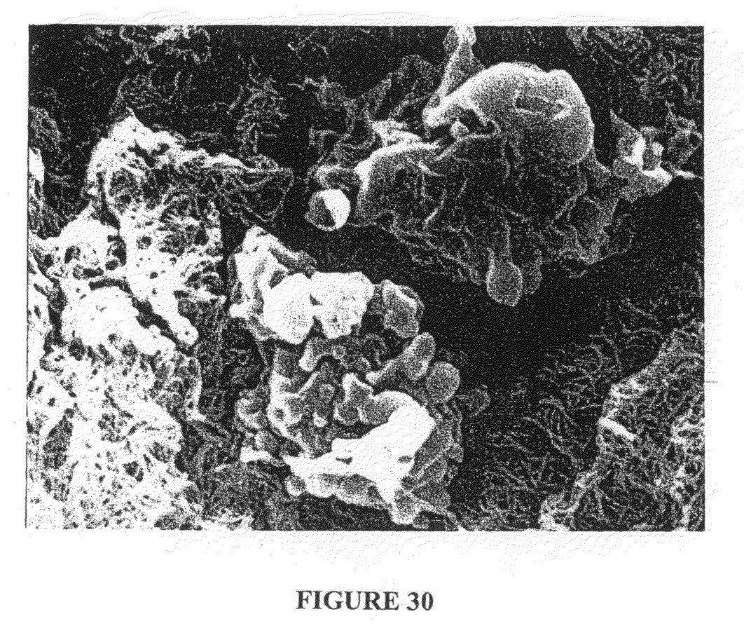

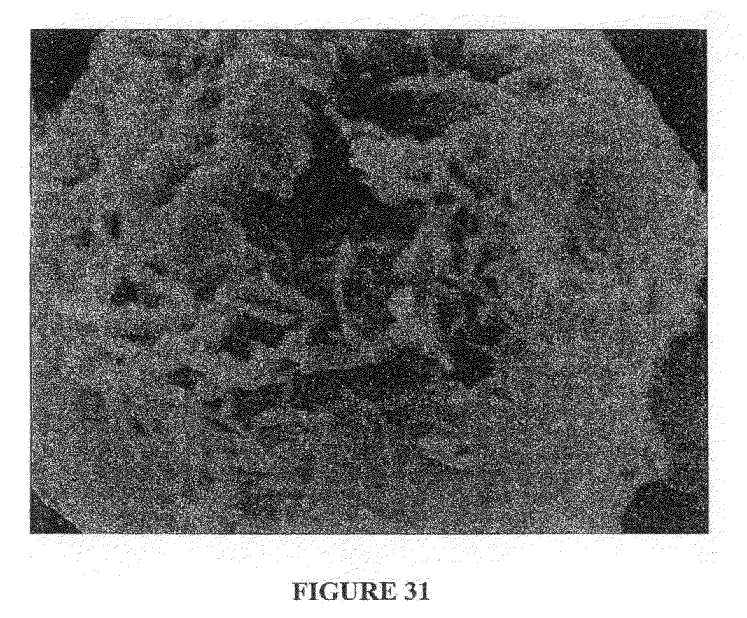

Referring to FIGS. 29-31, platelets from MEG-01 cells obtained following DMAT treatment form a fibrin clot when activated with 0.5 U/ml thrombin are described. FIG. 29 shows detail of the fibrin clot where aggregated platelets and specific activated platelets phenotype can be noticed. FIG. 30 shows platelets and fibrin net detail. FIG. 31 illustrates fibrin net detail from the clot.

All patents, published patent applications and published references noted herein are hereby incorporated by reference. The following is a representative listing of background articles.

Abelson, H. T., and Rabstein, L. S. (1970). Lymphosarcoma: virus-induced thymic-independent disease in mice. Cancer Res 30, 2213-2222.

Baatout, S. (1998). Phorbol esters: useful tools to study megakaryocyte differentiation. Hematol Cell Ther 40, 33-39.

Battinelli, E., Willoughby, S. R., Foxall, T., Valeri, C. R., and Loscalzo, J. (2001). Induction of platelet formation from megakaryocytoid cells by nitric oxide. Proc Natl Acad Sci USA 98, 14458-14463.

Battistutta, R., Mazzorana, M., Samo, S., Kazimierczuk, Z., Zanotti, G., and Pinna, L. A. (2005). Inspecting the structure-activity relationship of protein kinase CK2 inhibitors derived from tetrabromo-benzimidazole. Chem Biol 12, 1211-1219.

Borgers, M., Voipio-Pulkki, L., and Izumo, S. (2000). Apoptosis. Cardiovasc Res 45, 525-527.

Chen, J., De, S., Damron, D. S., Chen, W. S., Hay, N., and Byzova, T. V. (2004). Impaired platelet responses to thrombin and collagen in AKT-1-deficient mice. Blood 104, 1703-1710.

Clarke, M. C., Savill, J., Jones, D. B., Noble, B. S., and Brown, S. B. (2003). Compartmentalized megakaryocyte death generates functional platelets committed to caspase-independent death. J Cell Biol 160, 577-587.

Cozza, G., Bonvini, P., Zorzi, E., Poletto, G., Pagano, M. A., Samo, S., Donella-Deana, A., Zagotto, G., Rosolen, A., Pinna, L. A., et al. (2006). Identification of ellagic acid as potent inhibitor of protein kinase CK2: a successful example of a virtual screening application. J Med Chem 49, 2363-2366.

De Botton, S., Sabri, S., Daugas, E., Zermati, Y., Guidotti, J. E., Hermine, O., Kroemer, G., Vainchenker, W., and Debili, N. (2002). Platelet formation is the consequence of caspase activation within megakaryocytes. Blood 100, 1310-1317.

Deininger, M. W., and Druker, B. J. (2003). Specific targeted therapy of chronic myelogenous leukemia with imatinib. Pharmacol Rev 55, 401-423.

Druker, B. J., O'Brien, S. G., Cortes, J., and Radich, J. (2002). Chronic myelogenous leukemia. Hematology (Am Soc Hematol Educ Program), 111-135.

Fadeel, B., Zhivotovsky, B., and Orrenius, S. (1999). All along the watchtower: on the regulation of apoptosis regulators. FASEB J 13, 1647-1657.

Gewirtz, A. M. (1995). Megakaryocytopoiesis: the state of the art. Thromb Haemost 74, 204-209.

Glover, C. V. C., Sheltong, E. R., and Brutilag, D. L. (1983). Purification and Characterization of a Type I1 Casein Kinase from Drosophila melanogaster. J Biol Chem 258, 3258-3265.

Graham, K. G., and Litchfleld, D. W. (2000). The regulatory beta subunit of protein kinase CK2 mediates formation of tetrameric CK2 complexes. J Biol Chem 275, 5003-5010.

Hérichés, J. K., and Chambaz, E. M. (1998). Protein kinase CK2alpha is a target for the Abl and Bcr-Abl tyrosine kinases. Oncogene 17, 13-18.

Ishizaki, Y., Cheng, L., Mudge, A. W., and Raff, M. C. (1995). Programmed cell death by default in embryonic cells, fibroblasts, and cancer cells. Mol Biol Cell 6, 1443-1458.

Italiano J R., J. E., and Shivdasani, R. A. (2003). Megakaryocytes and beyond: the birth of platelets. J Thromb Haemost 1, 1174-1182.

Izeradjene, K., Douglas, L., Delaney, A., and Houghton, J. A. (2004). Influence of casein kinase II in tumor necrosis factor-related apoptosis-inducing ligand-induced apoptosis in human rhabdomyosarcoma cells. Clin Cancer Res 10, 6650-6660.

Izeradjene, K., Douglas, L., Delaney, A., and Houghton, J. A. (2005). Casein kinase II (CK2) enhances death-inducing signaling complex (DISC) activity in TRAIL-induced apoptosis in human colon carcinoma cell lines. Oncogene 24, 2050-2058.

Jiang, F., Jia, Y., and Cohen, I. (2002). Fibronectin- and protein kinase C-mediated activation of ERK/MAPK are essential for proplateletlike formation. Blood 99, 3579-3584.

Kaluzhny, Y., and Ravid, K. (2004). Role of apoptotic processes in platelet biogenesis. Acta Haematol 111, 67-77.

Kaluzhny, Y., Yu, G., Sun, S., Toselli, P. A., Nieswandt, B., Jackson, C. W., and Ravid, K. (2002). BclxL overexpression in megakaryocytes leads to impaired platelet fragmentation. Blood 100, 1670-1678.

Landesman-Bollag, E., Romieu-Mourez, R., Song, D. H., Sonenshein, G. E., Cardiff, R. D., and Seldin, D. C. (2001). Protein kinase CK2 in mammary gland tumorigenesis. Oncogene 20, 3247-3257.

Leist, M., and Jaattela, M. (2001). Four deaths and a funeral: from caspases to alternative mechanisms. Nat Rev Mol Cell Biol 2, 589-598.

Lionberger, J. M., Wilson, M. B., and Smithgall, T. E. (2000). Transformation of myeloid leukemia cells to cytokine independence by Bcr-Abl is suppressed by kinase-defective Hck. J Biol Chem 275, 18581-18585.

Liu, R. Y., Fan, C., Garcia, R., Jove, R., and Zuckerman, K. S. (1999). Constitutive activation of the JAK2/STAT5 signal transduction pathway correlates with growth factor independence of megakaryocytic leukemic cell lines. Blood 93, 2369-2379.

Mazur, E. M. (1987). Megakaryocytopoiesis and platelet production: a review. Exp Hematol, 340-350. McGahon, A., Bissonnette, R., Schmitt, M., Cotter, K. M., Green, D. R., and Cotter, T. G. (1994). BCR-ABL maintains resistance of chronic myelogenous leukemia cells to apoptotic cell death. Blood 83, 1179-1187.

Mishra, S., Pertz, V., Zhang, B., Kaur, P., Shimada, H., Groffen, J., Kazimierczuk, Z., Pinna, L. A., and Heisterkamp, N. (2006). Treatment of P190 Bcr/Abl lymphoblastic leukemia cells with inhibitors of the serine/threonine kinase CK2. Leukemia.

Mishra, S., Reichert, A., Cunnick, J., Senadheera, D., Hemmeryckx, B., Heisterkamp, N., and Groffen, J. (2003). Protein kinase CKIIalpha interacts with the Bcr moiety of Bcr/Abl and mediates proliferation of Bcr/Abl-expressing cells. Oncogene 22, 8255-8262.

Niefind, K., Guerra, B., Ermakowa, I., and Issinger, O. G. (2001). Crystal structure of human protein kinase CK2: insights into basic properties of the CK2 holoenzyme. Embo J 20, 5320. Nilsson, J., and Cleveland, J. L. (2003). Myc pathways provoking cell suicide and cancer. Oncogene 22, 9007-9021.

Ogura, M., Morishima, Y., Ohno, R., Kato, Y., Hirabayashi, N., Nagura, H., and Saito, H. (1985). Establishment of a novel human megakaryoblastic leukemia cell line, MEG-01, with positive Philadelphia chromosome. Blood 66, 1384-1392.

Ogura, M., Morishima, Y., Okumura, M.; Hotta, T., Takamoto, S., Ohno, R., Hirabayashi, N., Nagura, H., and Saito, H. (1988). Functional and morphological differentiation induction of a human megakaryoblastic leukemia cell line (MEG-01s) by phorbol diesters. Blood 72, 49-60.

Padmanabha, R., and Glover, C. V. C. (1987). Casein kinase II of yeast contains two distinct a polypeptides and an unusually large b subunit. J Biol Chem 262, 1829-1835.

Pagano, M. A., Andrzejewska, M., Ruzzene, M., Sarno, S., Cesaro, L., Bain, J., Elliott, M., Meggio, F., Kazimierczuk, Z., and Pinna, L. A. (2004a). Optimization of protein kinase CK2 inhibitors derived from 4,5,6,7-tetrabromobenzimidazole. J Med Chem 47, 6239-6247.

Pagano, M. A., Cesaro, L., Megglo, F., and Pinna, L. A. (2006). Protein kinase CK2: a newcomer in the ‘druggable kinome’. Biochem Soc Trans 34, 1303-1306.

Pagano, M. A., Meggio, F., Ruzzene, M., Andrzejewska, M., Kazimierczuk, Z., and Pinna, L. A. (2004b). 2-Dimethylamino-4,5,6,7-tetrabromo-1H-benzimidazole: a novel powerful and selective inhibitor of protein kinase CK2. Biochem Biophys Res Commun 321, 1040-1044.

Patel, S. R., Hartwig, J. H., and Italiano, J. E. J. (2005). The biogenesis of platelets from megakaryocyte proplatelets. J Clin Invest 115, 3348-3354.

Pechkova, E., Zanotti, G., and Nicolini, C. (2003). Three-dimensional atomic structure of a catalytic subunit mutant of human protein kinase CK2. Acta Crystallogr 59, 2133.

Phan-Dinh-Tuy, F., Henry, J., Boucheix, C., Perrot, J. Y., Rosenfeld, C., and Kahn, A. (1985). Protein kinases in human leukemic cells. Am J Hematol 19, 209-218.

Piazza, F. A., Ruzzene, M., Gurrieri, C., Montini, B., Bonanni, L., Chioetto, G., Di Maira, G., Barbon, F., Cabrelle, A., Zambello, R., et al. (2006). Multiple myeloma cell survival relies on high activity of protein kinase CK2. Blood.

Pinna, L. A. (1990). Casein kinase 2: an ‘eminence grise’ in cellular regulation. Biochem Biophys Acta 1054, 267-284.

Pinna, L. A. (1997). Protein kinase CK2. J Biochem Cell Biol 29, 551-554.

Pinna, L. A. (2002). Protein kinase CK2: a challenge to canons. J Cell Sci 115, 3873-3878.

Pinna, L. A. (2003). The raison d'etre of constitutively active protein kinases: the lesson of CK2. Acc Chem Res 36, 378-384.

Rowley, J. D. (1973). Letter: A new consistent chromosomal abnormality in chronic myelogenous leukaemia identified by quinacrine fluorescence and Giemsa staining. Nature 243, 290-293.

Ruzzene, M., Penzo, D., and Pinna, L. A. (2002). Protein kinase CK2 inhibitor 4,5,6,7-tetrabromobenzotriazole (TBB) induces apoptosis and caspase-dependent degradation of haematopoietic lineage cell-specific protein 1 (HS1) in Jurkat cells. Biochem J 364, 41-47.

Saraste, A., and Pulkki, K. (2000). Morphologic and biochemical hallmarks of apoptosis. 45, 528-537.

Sarno, S., Ruzzene, M., Frascella, P., Pagano, M. A., Meggio, F., Zambon, A., Mazzorana, M., Di Maira, G., Lucchini, V., and Pinna, L. A. (2005a). Development and exploitation of CK2 inhibitors. Mol Cell Biochem 274, 69-76.

Sarno, S., Salvi, M., Battistutta, R., Zanotti, G., and Pinna, L. A. (2005b). Features and potentials of ATP-site directed CK2 inhibitors. Biochim Biophys Acta 1754, 263-270.

Schmidt-Spaniol, I., Grimm, B., and Issinger, O. G. (1993). Subcellular localization of protein kinase CK-2 alpha- and beta-subunits in synchronized cells from primary human fibroblasts and established cell lines. Cell Mol Biol Res 39, 761-772.

Seldin, D. C., Landesman-Bollag, E., Farago, M., Currier, N., Lou, D., and Dominguez, I. (2005). CK2 as a positive regulator of Wnt signalling and tumourigenesis. Mol Cell Biochem 274, 63-67.

Shattil, S. J., Cunningham, M., and Hoxie, J. A. (1987). Detection of activated platelets in whole blood using activation-dependent monoclonal antibodies and flow cytometry. Blood 70, 307-315.

Shattil, S. J., Hoxie, J. A., Cunningham, M., and Brass, L. F. (1985). Changes in the platelet membrane glycoprotein IIb.IIIa complex during platelet activation. J Biol Chem 260, 11107-11114.

Shattil, S. J., and Leavitt, A. D. (2001). All in the family: primary megakaryocytes for studies of platelet alphaIIbbeta3 signaling. Thromb Haemost 86, 259-265.

Slaton, J. W., Unger, G. M., Sloper, D. T., Davis, A. T., and Ahmed, K. (2004). Induction of apoptosis by antisense CK2 in human prostate cancer xenograft model. Mol Cancer Res 2, 712-721.

Stigare, J., Buddelmeijer, N., Pigon, A., and Egyhazi, E. (1993). A majority of casein kinase II alpha subunit is tightly bound to intranuclear components but not to the beta subunit. Mol Cell Biochem 129, 77-85.

Takeuchi, K., Ogura, M., Saito, H., Satoh, M., and Takeuchi, M. (1991). Production of platelet-like particles by a human megakaryoblastic leukemia cell line (MEG-01). Exp Cell Res 193, 223-226.

Takeuchi, K., Satoh, M., Kuno, H., Yoshida, T., Kondo, H., and Takeuchi, M. (1998). Platelet-like particle formation in the human megakaryoblastic leukaemia cell lines, MEG-01 and MEG-01s. Br J Haematol 100, 436-444.

Unger, G. M., Davis, A. T., Slaton, J. W., and Ahmed, K. (2004). Protein kinase CK2 as regulator of cell survival: implications for cancer therapy. Curr Cancer Drug Targets 4, 77-84.

Wang, H., Davis, A., Yu, S., and Ahmed, K. (2001). Response of cancer cells to molecular interruption of the CK2 signal. Mol Cell Biochem 227, 167-174.

Wertheim, J. A., Forsythe, K., Druker, B. J., Hammer, D., Boettiger, D., and Pear, W. S. (2002). BCR-ABL-induced adhesion defects are tyrosine kinase-independent. Blood 99, 4122-4130.

Yamazaki, Y., Sanokawa, R., Fujita, Y., Zhou, D., Kawasaki, K., Tanaka, H., Komatsu, T., Nagasawa, T., and Oka, S. (1999). Cytoplasmic elongation and rupture in megakaryoblastic leukemia cells via activation of adhesion and motility by staurosporine on fibronectin-bound substratum. J Cell Physiol 179, 179-192.

Zauli, G., Vitale, M., Falcieri, E., Gibellini, D., Bassini, A., Celeghini, C., Columbaro, M., and Capitani, S. (1997). In vitro senescence and apoptotic cell death of human megakaryocytes. Blood 90, 2234-2243.

Zunino, R., U, Q., Rose, S. D., Romero-Benitez, M. M., Lejen, T., Brandan, N. C., and Triffaro, J. M. (2001). Expression of scinderin in megakaryoblastic leukemia cells induces differentiation, maturation, and apoptosis with release of plateletlike particles and inhibits proliferation and tumorigenesis. Blood 98, 2210-2219.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A method for treating myeloproliferative disorders, comprising:
selectively inhibiting CK2α activity.

2. The method of claim 1 wherein selectively inhibiting CK2α activity comprises administering an amount of a CK2α selective inhibitor effective to inhibit thrombocytopoiesis.

3. The method of claim 2 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

4. The method of claim 1 wherein the disorder is chronic myelogenous leukemia.

5. The method of claim 1 wherein the disorder is thrombocytosis.

6. The method of claim 3 wherein the CK2α selective inhibitor is DMAT.

7. The method of claim 6 wherein DMAT is used in a concentration of from 0.1 μM to 1,000 μM.

8. The method of claim 7 wherein DMAT is used in a concentration of from about 1 μM to about 100 μM.

9. The method of claim 8 wherein DMAT is used in a concentration of from about 10 μM to about 50 μM.

10. The method of claim 3 wherein the CK2α selective inhibitor is TBBt.

11. The method of claim 10 wherein TBBt is used in a concentration of from 0.1 μM to 1,000 μM.

12. The method of claim 11 wherein TBBt is used in a concentration of from about 1 μM to about 150 μM.

13. The method of claim 12 wherein TBBt is used in a concentration of from about 15 μM to about 75 μM.

14. The method of claim 2 wherein the CK2α selective inhibitor is administered for a period of from about 1 to about 14 days.

15. The method of claim 14 wherein the CK2α selective inhibitor is administered for a period of from about 3 to about 7 days.

16. The method of claim 2 wherein the CK2α selective inhibitor is administered in a dosage unit of from about 0.001 to about 100 mg/kg.

17. The method of claim 16 wherein the CK2α selective inhibitor is administered in a dosage unit of from about 0.01 to about 50 mg/kg.

18. The method of claim 17 wherein the CK2α selective inhibitor is administered in a dosage unit of from about 0.05 to about 20 mg/kg.

19. A pharmaceutical composition comprising:
a CK2α selective inhibitor selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii); and
a pharmaceutically acceptable carrier.

20. The composition of claim 19 wherein the CK2α selective inhibitor is TBBt.

21. The composition of claim 19 wherein the CK2α selective inhibitor is DMAT.

22. A method for inhibiting hematological malignancies, comprising:
selectively inhibiting CK2α activity.

23. The method of claim 22 wherein selectively inhibiting CK2α activity comprises administering an amount of a CK2α selective inhibitor effective to inhibit thrombocytopoiesis.

24. The method of claim 23 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

25. The method of claim 22 wherein the hematological malignancy is chronic myelogenous leukemia.

26. A method for inducing maturation of malignant megakaryoblasts, comprising:
selectively inhibiting CK2α activity.

27. The method of claim 26 wherein selectively inhibiting CK2α activity comprises administering an amount of a CK2α selective inhibitor effective to inhibit thrombocytopoiesis.

28. The method of claim 27 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

29. The method of claim 26 whereby uncontrolled platelet production otherwise occurring is reduced.

30. A method for inducing thrombocytosis, comprising:
selectively inhibiting CK2α activity.

31. The method of claim 30 wherein selectively inhibiting CK2α activity comprises administering an amount of a CK2α selective inhibitor effective to inhibit thrombocytopoiesis.

32. The method of claim 31 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

33. The method of claim 30 wherein inhibiting CK2α activity inhibits thrombocytopolesis and therefore reduces production of platelets from megakaryocytes.

34. A method for reducing platelet production occurring from malignant megakaryoblasts, comprising:
selectively inhibiting CK2α activity.

35. The method of claim 34 wherein selectively inhibiting CK2α activity comprises administering an amount of a CK2α selective inhibitor effective to inhibit thrombocytopoiesis.

36. The method of claim 35 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).

37. A method for inducing thrombocytopoiesis, comprising:
selectively inhibiting CK2α activity.

38. The method of claim 37 wherein selectively inhibiting CK2α activity comprises administering an amount of a CK2α selective inhibitor effective to inhibit thrombocytopoiesis.

39. The method of claim 38 wherein the CK2α selective inhibitor is selected from the group consisting of (i) 4,5,6,7-Tetrabromobenzotriazole (TBBt), (ii) 2-Dimethylamino-4,5,6,7-tetrabromo-1H-benzimidazole (DMAT), and combinations of (i) and (ii).
